SEQUENCE LISTING (24 sequences)

SEQ ID NO: 1 - protein, 664 aa, Influenza virus
MFLLLATITAITACQAERELICIVQRVNESFSLHSGFGGNVYSMKTEPMTGFTNVTKGASVINQKDWIGFGDSRTDLTNDQFPASSDVPLAVAKKFRSLSGASLMLSAFGPPGKVDYLYQGCGKEKVFYEGVNWSPEAGIDCFGSNWTQTKKDFYSRIYEAARSSTCMTLVNSLDTKISSTTATAGTASSCSSSWMKSPLWYAESSVNPGAKPQVCGTEQSATFTLPTSFGIYKCNKHVVQLCYFVYENKAKFNTFGCGDYYQNYYDGNGNLIGGMDNRVAAYRGIANAGVKIECPSKILNPGTYSIKSTPRFLLVPKRSYCFDTDGGYPIQVVQSEWSASRRSDNATEEACLQTEGCIFIKKTTPYVGEADDNHGDIEMRQLLSGLGNNDTVCVSQSGYTKGETPFVKDYLSPPKYGRCQLKTDSGRIPTLPSGLIIPQAGTDSLMRTLTPATRIFGIDDLIFGLLFVGFVAGGVAGGYFWGRSNGGGGGASVSSTQAGFDKIGKDIQQLRNDTNAAIEGFNGRIAHDEQAIKNLAKEIEDARAEALVGELGIIRSLIVANISMNLKESLYELANQITKRGGGIAQEAGPGCWYVDSENCDASCKEYIFNFNGSATVPTLRPVDTKVVITSDPYYLGSTIALCLLGLVAIAASVGVIWICCKK

SEQ ID NO: 2 - DNA, 220 bp, Influenza virus
  misc_feature (1)..(220): Synthetic cDNA hemagglutinin packaging construct based upon influenza A hemagglutinin segment.
  misc_feature (86)..(86): n is a, t, c, or g, position of insertion of open reading frame sequence encoding heterologous protein
agcaaaagcaggggaaaataaaaacaaccaaactgaaggcaaaactactggtcctgttatatgcatttgtagctacagcggccgcncctgcaggctggtgcttttggtctccctgggggcaatcagtttctggatgtgttctaatgggtctttgcagtgcagaatatgcatctgagattaggatttcagaaatataaggaaaaacacccttgtttctact

SEQ ID NO: 3 - DNA, 333 bp, Influenza virus
  misc_feature (1)..(333): Synthetic cDNA neuraminidase packaging construct based upon influenza A neuraminidase segment.
  misc_feature (139)..(139): n is a, t, c, or g, position of insertion of open reading frame sequence encoding heterologous protein
agcaaaagcaggagtttaattgaatccaaaccagaaaataataaccattgggtcaatctgtttggtagtcggaataattagcctaatattgcaaataggaaatataatctcaatttggattagccattcagcggccgcncctgcaggtgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 4 - DNA, 2214 bp, Influenza virus
  misc_feature (1)..(2214): Synthetic cDNA construct encoding chimeric influenza A viral hemagglutinin segment expressing influenza D hemagglutinin esterase fusion protein
agcaaaagcaggggaaaataaaaacaaccaaactgaaggcaaaactactggtcctgttatatgcatttgtagctacagcggccgcatgtttttgcttctagcaacaattacagcaataactgcttgccaagcagaaagagaactgatatgcatagtgcaaagagtgaatgaaagcttctctcttcactctggatttggaggaaatgtttacagcatgaagactgagccaatgactggattcacaaacgtgaccaaaggtgctagtgtcatcaaccaaaaagactggattggattcggagattcaagaacagacttgactaatgatcagtttccagcgtcttcagatgttccattggcagtggcgaagaagtttcggtcattgtcaggggcttcgttgatgttgtcagcttttgggcctcctggcaaggttgactacctctatcaaggatgtgggaaagaaaaagtattttatgaaggggtaaactggtcccctgaggcaggaattgattgctttggatcaaactggactcagacaaagaaggacttctattcgaggatatatgaagctgctagaagcagcacatgcatgactcttgtaaattctcttgacaccaagatatcatcaacaacagccacggctggaaccgcatcttcttgttcttcaagttggatgaaaagcccgttgtggtatgcagaatcttctgttaatcctggagctaaacctcaagtttgtgggactgagcaatcggcaacttttactttgccgacaagcttcggaatttacaaatgcaacaagcatgtagtgcagctttgttactttgtgtacgaaaacaaagcaaaatttaacacttttggctgtggagattattaccaaaattactatgatgggaatggaaacctgatagggggaatggataacagagtggcagcatacagaggaatagcaaacgctggagttaaaattgaatgtccttccaaaatcttgaaccctgggacttacagcattaaatcaacaccaagattccttctagtaccaaaaaggtcatactgcttcgacactgatggagggtaccctatacaagtagttcaatctgagtggtcagcttcacgaagatcagataatgccacagaagaagcatgcctacaaacagaaggatgtattttcatcaaaaagacaaccccttatgtaggagaagcagatgacaaccatggagacattgagatgaggcaactcttgagtgggcttggcaacaacgacacagtgtgcgtttcccaaagtggatacacaaaaggagagaccccttttgtaaaggattatttgagtcctcccaagtatggcagatgtcagttgaaaactgacagtggaagaatcccaactctaccttctgggttgataataccgcaagcagggactgactctttaatgagaactttgacgccagcaacaaggatcttcggaatagatgacttaatcttcgggcttttattcgtggggtttgtcgcaggaggggtcgcaggaggttacttctggggaagatcaaatggagggggtggtggtgcctcggtgagcagtacgcaggctggatttgacaaaatcggaaaagatatacagcagcttcggaatgacacaaatgcagcaattgaaggcttcaacgggagaattgcccatgatgagcaagccattaagaatttggcaaaagaaatcgaagatgcaagggcagaggctttggtaggggaacttggtataataagatccctcatagttgccaacataagcatgaatctaaaagaatctttatatgaactagcaaaccaaataacaaaaagaggaggaggaattgcacaagaagcaggcccagggtgttggtatgttgactccgaaaactgtgatgcaagctgcaaagagtacattttcaacttcaatggaagtgccactgtccccacattgaggccagttgacaccaaggttgtaataacatcggatccttattacttgggttcgaccatagctctctgtcttttggggctggtggcgattgctgcttctgttggtgtgatttggatctgttgcaagaaatagcctgcaggctggtgcttttggtctccctgggggcaatcagtttctggatgtgttctaatgggtctttgcagtgcagaatatgcatctgagattaggatttcagaaatataaggaaaaacacccttgtttctact

SEQ ID NO: 5 - DNA, 1410 bp, Influenza A virus
  misc_feature (1)..(1410): Synthetic cDNA construct encoding for influenza A NA segment
atgaatccaaaccaaaagataataaccattggttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtgtaagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacaatatattgagaacacaagagtctgaatgtgcatgtgtaaatggttcttgctttactgtaatgaccgatggaccaagtaatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaagggttttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaagtaa

SEQ ID NO: 6 - protein, 469 aa, Influenza A virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 7 - DNA, 704 bp, Influenza virus
  misc_feature (1)..(704): Synthetic cDNA chimera of influenza A NA packaging construct and Porcine Reproductive and Respiratory Syndrome virus nucleocapsid coding sequences
agcaaaagcaggagtttaattgaatccaaaccagaaaataataaccattgggtcaatctgtttggtagtcggaataattagcctaatattgcaaataggaaatataatctcaatttggattagccattcagcggccgcatgccaaataacaacggcaggcagcagaagaaaaagaaaggggacggccagccagtcaatcagctgtgccaaatgttgggcaggatcatcgcccagcaaaaccagtccagaggtaagggaccggggaagaaaagtaagaagaaaagcccggagaagccccattttcctctcgcgactgaagatgacgttagacatcacttcacccctagtgagcggcaattgtgtctgtcgtcaatccagactgcctttaaccaaggcgctggaacttgtaccctgtcggattcagggagaataagttacgctgtggagtttagtttgcctacgcatcatactgtgcgcctaattcgcgtcacagcatcaccctcagcatgacctgcaggtgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 8 - DNA, 2483 bp, Influenza virus
  misc_feature (1)..(2483): Synthetic cDNA chimera of influenza A NA packaging construct and Porcine epidemic diarrhea virus spike protein coding sequences
agcaaaagcaggagtttaattgaatccaaaccagaaaataataaccattgggtcaatctgtttggtagtcggaataattagcctaatattgcaaataggaaatataatctcaatttggattagccattcagcggccgcatgaagtctctcacctatttttggctctttctgcccgtgctgtcaacactctcactgcctcaggatgtcacccgctgctccgctaacaccaacttccgccggttctttagcaagtttaatgtgcaggctcctgctgtggtcgtgctcggaggatacctgccaatcggagagaatcagggggtgaactccacctggtattgcgctggacagcacccaacagctagcggagtccatgggatcttcgtgtctcacattagggggggccatggcttcgagatcggaatttcacaggaaccttttgacccctccggctaccagctgtatctccacaaggccaccaacggaaataccaacgctacagcccgactgcgcatctgtcagttccccagcattaagaccctggggccaacagccaacaatgatgtgaccacaggcagaaattgcctgttcaacaaagctatcccagcccacatgtccgagcatagcgtcgtgggcattacctgggacaacgatcgcgtcacagtgttctccgacaagatctactatttctactttaaaaacgattggagccgggtggccaccaagtgttataattccggagggtgcgctatgcagtacgtctatgagccaacctactatatgctgaacgtgacaagcgccggcgaagacggaatttcttaccagccttgtaccgccaattgcatcggctatgccgctaacgtgttcgccacagagccaaatggacacatccctgaagggttcagctttaacaattggtttctgctctctaacgactcaaccctggtgcatggaaaggtcgtgtccaatcagccactgctcgtgaactgtctgctcgccatccctaaaatctacgggctgggccagttctttagcttcaaccagaccatcgacggggtctgcaatggagctgctgtgcagcgagctcctgaggccctgcgcttcaatatcaacgatacctcagtcattctggccgaaggctccatcgtgctccacaccgctctggggacaaacttctccttcgtgtgcagcaattccagcaacccccatctcgctaccttcgccatccctctgggagctacacaggtcccctactattgcttcctgaaggtggacacctacaactccacagtctataaattcctggccgtgctcccaccaaccgtccgcgagatcgtgattacaaagtacggcgacgtgtacgtgaacggattcgggtacctgcacctcgggctgctcgatgccgtgaccatcaacttcaccggccatggaaccgacgatgacgtgagcggattctggacaattgcctctaccaactttgtggacgccctgatcgaggtgcagggaaccgccatccagaggattctctactgtgatgaccccgtctcccagctgaaatgcagccaggtcgccttcgacctcgatgacggcttttacccaatttcttcaagaaacctgctctcacacgagcagccaatctccttcgtgaccctgccttctttcaatgatcactcatttgtcaacatcacagtgtccgccagctttggcggacattcaggggccaacctgattgcttccgacaccacaatcaatggcttctccagcttttgcgtggacacacggcagttcaccatctcactgttttacaatgtcaccaactcctacggctacgtgtccaagtcacaggacagcaactgccccttcacactccagtctgtcaatgattacctgtccttcagcaaattttgcgtgtccacctccctgctcgctagcgcctgcacaatcgacctgttcggctaccctgagtttgggagcggcgtgaagttcacctctctgtatttccagtttacaaagggcgagctcattaccggaacccctaaacccctggaaggcgtcacagacgtgtccttcatgaccctggacgtgtgcaccaagtacacaatctatggattcaaaggagaggggatcattaccctcacaaactcttcatttctggccggcgtgtactatacctccgacagcggacagctgctcgccttcaagaacgtgacctccggggctgtctacagcgtgacaccatgctctttttcagagcaggccgcttatgtggatgacgatatcgtcggagtgattcctgcaggtgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 9 - DNA, 1034 bp, Influenza virus
  misc_feature (1)..(1034): Synthetic cDNA chimera of influenza A NA packaging construct and porcine circovirus capsid protein coding sequences
agcaaaagcaggagtttaattgaatccaaaccagaaaataataaccattgggtcaatctgtttggtagtcggaataattagcctaatattgcaaataggaaatataatctcaatttggattagccattcagcggccgcatgacgtatccaaggaggcgttaccggagaagaagacaccgcccccgcagccatcttggccagatcctccgccgccgcccctggctcgtccacccccgccaccgttaccgctggagaaggaaaaatggcatcttcaacacccgcctatcccgcaccttcggatatactatcaagcgaaccacagtcagaacgccctcctgggcggtggacatgatgagattcaatattaattactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtcctagattccactattgattacttccaaccaaacaacaaaagaaaccagctgtggctgagactacaaactgctggaaatgtagaccacgtaggcctcggcactgcgttcgaaaacagtatatacgaccaggaatacaatatccgtgtaaccatgtatgtacaattcagagaatttaatcttaaagaccccccacttaacccttaacctgcaggtgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 10 - DNA, 1052 bp, Influenza virus
  misc_feature (1)..(1052): Synthetic cDNA chimera of influenza A NA packaging construct and reporter protein green fluorescent protein coding sequences
agcaaaagcaggagtttaattgaatccaaaccagaaaataataaccattgggtcaatctgtttggtagtcggaataattagcctaatattgcaaataggaaatataatctcaatttggattagccattcagcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctgcaggtgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 11 - DNA, 2274 bp, Influenza A virus
  misc_feature (1)..(2274): Synthetic cDNA construct coding for influenza A PB1 viral segment
atggatgtcaatccgactctacttttcctaaaaattccagcgcaaaatgccataagcaccacattcccttatactggagatcctccatacagccatggaacaggaacaggatacaccatggacacagtaaacagaacacaccaatactcagaaaagggaaagtggacgacaaacacagagactggtgcaccccagctcaacccgattgatggaccactacctgaggataatgaaccaagtgggtatgcacaaacagactgtgttctagaggctatggctttccttgaagaatcccacccaggaatatttgagaattcatgccttgaaacaatggaagttgttcaacaaacaagggtagataaactaactcaaggtcgccagacttatgattggacattaaacagaaatcaaccggcagcaactgcattggccaacaccatagaagtctttagatcgaatggcctaacagctaatgagtcaggaaggctaatagatttcttaaaggatgtaatggaatcaatgaacaaagaggaaatagagataacaacccactttcaaagaaaaaggagagtaagagacaacatgaccaagaagatggtcacgcaaagaacaatagggaagaaaaaacaaagactgaataagagaggctatctaataagagcactgacattaaatacgatgaccaaagatgcagagagaggcaagttaaaaagaagggctatcgcaacacctgggatgcagattagaggtttcgtatactttgttgaaactttagctaggagcatttgcgaaaagcttgaacagtctgggctcccagtagggggcaatgaaaagaaggccaaactggcaaatgttgtgagaaagatgatgactaattcacaagacacagagatttctttcacaatcactggggacaacactaagtggaatgaaaatcaaaatcctcgaatgttcctggcgatgattacatatatcaccagaaatcaacccgagtggttcagaaacatcctgagcatggcacccataatgttctcaaacaaaatggcaagactagggaaagggtacatgttcgagagtaaaagaatgaagattcgaacacaaataccagcagaaatgctagcaagcattgacctgaagtacttcaatgaatcaacaaagaagaaaattgagaaaataaggcctcttctaatagatggcacagcatcactgagtcctgggatgatgatgggcatgttcaacatgctaagtacggtcttgggagtctcgatactgaatcttggacaaaagaaatacaccaagacaatatactggtgggatgggctccaatcatccgacgattttgctctcatagtgaatgcaccaaaccatgagggaatacaagcaggagtggacagattctacaggacctgcaagttagtgggaatcaacatgagcaaaaagaagtcctatataaataagacagggacatttgaattcacaagctttttttatcgctatggatttgtggctaattttagcatggagctacccagctttggagtgtctggagtaaatgaatcagctgacatgagtattggagtaacagtgataaagaacaacatgataaacaatgaccttggacctgcaacggcccagatggctcttcaattgttcatcaaagactacagatacacatataggtgccataggggagacacacaaattcagacgagaagatcatttgagttaaagaagctgtgggatcaaacccaatcaaaagtagggctattagtatcagatggaggaccaaacttatacaatatacggaatcttcacattcctgaagtctgcttaaaatgggagctaatggatgatgattatcggggaagactttgtaatcccctgaatccctttgtcagtcataaagagattgattctgtaaacaatgctgtggtaatgccagcccatggtccagccaaaagcatggaatatgatgccgttgcaactacacattcctggattcccaagaggaatcgttctattctcaacacaagccaaaggggaattcttgaggatgaacagatgtaccagaagtgctgcaatctattcgagaaatttttccctagcagttcatataggagaccggttggaatttctagcatggtggaggccatggtgtctagggcccggattgatgccagggtcgacttcgagtctggacggatcaagaaagaagagttctctgagatcatgaagatctgttccaccattgaagaactcagacggcaaaaataa

SEQ ID NO: 12 - protein, 757 aa, Influenza A virus
MDVNPTLLFLKIPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVVQQTRVDKLTQGRQTYDWTLNRNQPAATALANTIEVFRSNGLTANESGRLIDFLKDVMESMNKEEIEITTHFQRKRRVRDNMTKKMVTQRTIGKKKQRLNKRGYLIRALTLNTMTKDAERGKLKRRAIATPGMQIRGFVYFVETLARSICEKLEQSGLPVGGNEKKAKLANVVRKMMTNSQDTEISFTITGDNTKWNENQNPRMFLAMITYITRNQPEWFRNILSMAPIMFSNKMARLGKGYMFESKRMKIRTQIPAEMLASIDLKYFNESTKKKIEKIRPLLIDGTASLSPGMMMGMFNMLSTVLGVSILNLGQKKYTKTIYWWDGLQSSDDFALIVNAPNHEGIQAGVDRFYRTCKLVGINMSKKKSYINKTGTFEFTSFFYRYGFVANFSMELPSFGVSGVNESADMSIGVTVIKNNMINNDLGPATAQMALQLFIKDYRYTYRCHRGDTQIQTRRSFELKKLWDQTQSKVGLLVSDGGPNLYNIRNLHIPEVCLKWELMDDDYRGRLCNPLNPFVSHKEIDSVNNAVVMPAHGPAKSMEYDAVATTHSWIPKRNRSILNTSQRGILEDEQMYQKCCNLFEKFFPSSSYRRPVGISSMVEAMVSRARIDARVDFESGRIKKEEFSEIMKICSTIEELRRQK

SEQ ID NO: 13 - DNA, 2280 bp, Influenza A virus
  misc_feature (1)..(2280): Synthetic cDNA construct coding for influenza A PB2 viral segment
atggagagaataaaagaactgagagatctaatgtcgcagtcccgcactcgcgagatactcactaagaccactgtggaccatatggccataatcaaaaagtacacatcaggaaggcaagagaagaaccccgcactcagaatgaagtggatgatggcaatgagatacccaattacagcagacaagagaataatggacatgattccagagaggaatgaacaaggacaaaccctctggagcaaaacaaacgatgctggatcagaccgagtgatggtatcacctctggccgtaacatggtggaataggaatggcccaacaacaagtacagttcattaccctaaggtatataaaacttatttcgaaaaggtcgaaaggttgaaacatggtaccttcggccctgtccacttcagaaatcaagttaaaataaggaggagagttgatacaaaccctggccatgcagatctcagtgccaaggaggcacaggatgtgattatggaagttgttttcccaaatgaagtgggggcaagaatactgacatcagagtcacagctggcaataacaaaagagaagaaagaagagctccaggattgtaaaattgctcccttgatggtggcgtacatgctagaaagagaattggtccgtaaaacaaggtttctcccagtagccggcggaacaggcagtgtttatattgaagtgttgcacttaacccaagggacgtgctgggagcagatgtacactccaggaggagaagtgagaaatgatgatgttgaccaaagtttgattatcgctgctagaaacatagtaagaagagcagcagtgtcagcagacccattagcatctctcttggaaatgtgccacagcacacagattggaggagtaaggatggtggacatccttagacagaatccaactgaggaacaagccgtagacatatgcaaggcagcaatagggttgaggattagctcatctttcagttttggtgggttcactttcaaaaggacaagcggatcatcagtcaagaaagaagaagaagtgctaacgggcaacctccaaacactgaaaataagagtacatgaagggtatgaagaattcacaatggttgggagaagagcaacagctattctcagaaaggcaaccaggagattgatccagttgatagtaagcgggagagacgagcagtcaattgctgaggcaataattgtggccatggtattctcacaggaggattgcatgatcaaggcagttaggggcgatctgaactttgtcaatagggcaaaccagcgactgaaccccatgcaccaactcttgaggcatttccaaaaagatgcaaaagtgcttttccagaactggggaattgaatccatcgacaatgtgatgggaatgatcggaatactgcccgacatgaccccaagcacggagatgtcgctgagagggataagagtcagcaaaatgggagtagatgaatactccagcacggagagagtggtagtgagtattgaccgatttttaagggttagagatcaaagagggaacgtactattgtctcccgaagaagtcagtgaaacgcaaggaactgagaagttgacaataacttattcgtcatcaatgatgtgggagatcaatggccctgagtcagtgctagtcaacacttatcaatggataatcaggaactgggaaattgtgaaaattcaatggtcacaagatcccacaatgttatacaacaaaatggaatttgaaccatttcagtctcttgtccctaaggcaaccagaagccggtacagtggattcgtaaggacactgttccagcaaatgcgggatgtgcttgggacatttgacactgtccaaataataaaacttctcccctttgctgctgccccaccagaacagagtaggatgcaattttcctcattgactgtgaatgtgagaggatcagggttgaggatactggtaagaggcaattctccagtattcaattacaacaaggcaaccaaacgacttacagttcttggaaaggatgcaggtgcattgactgaagatccagatgaaggcacatctggggtggagtctgctgtcctgagaggatttctcattttgggcaaagaagacaagagatatggcccagcattaagcatcaatgaactgagcaatcttgcaaaaggagagaaggctaatgtgctaattgggcaaggggacgtagtgttggtaatgaaacgaaaacgggactctagcatacttactgacagccagacagcgaccaaaagaattcggatggccatcaattag

SEQ ID NO: 14 - protein, 759 aa, Influenza A virus
MERIKELRDLMSQSRTREILTKTTVDHMAIIKKYTSGRQEKNPALRMKWMMAMRYPITADKRIMDMIPERNEQGQTLWSKTNDAGSDRVMVSPLAVTWWNRNGPTTSTVHYPKVYKTYFEKVERLKHGTFGPVHFRNQVKIRRRVDTNPGHADLSAKEAQDVIMEVVFPNEVGARILTSESQLAITKEKKEELQDCKIAPLMVAYMLERELVRKTRFLPVAGGTGSVYIEVLHLTQGTCWEQMYTPGGEVRNDDVDQSLIIAARNIVRRAAVSADPLASLLEMCHSTQIGGVRMVDILRQNPTEEQAVDICKAAIGLRISSSFSFGGFTFKRTSGSSVKKEEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIESIDNVMGMIGILPDMTPSTEMSLRGIRVSKMGVDEYSSTERVVVSIDRFLRVRDQRGNVLLSPEEVSETQGTEKLTITYSSSMMWEINGPESVLVNTYQWIIRNWEIVKIQWSQDPTMLYNKMEFEPFQSLVPKATRSRYSGFVRTLFQQMRDVLGTFDTVQIIKLLPFAAAPPEQSRMQFSSLTVNVRGSGLRILVRGNSPVFNYNKATKRLTVLGKDAGALTEDPDEGTSGVESAVLRGFLILGKEDKRYGPALSINELSNLAKGEKANVLIGQGDVVLVMKRKRDSSILTDSQTATKRIRMAIN

SEQ ID NO: 15 - DNA, 2151 bp, Influenza A virus
  misc_feature (1)..(2151): Synthetic cDNA construct coding for influenza A PA viral segment
atggaagactttgtgcgacaatgcttcaatccaatgatcgtcgagcttgcggaaaaggcaatgaaagaatatggggaagatccgaaaatcgaaactaacaagtttgctgcaatatgcacacatttggaagtttgtttcatgtattcggatttccatttcatcgacgaacggggtgaatcaataattgtagaatctggtgacccgaatgcactattgaagcaccgatttgagataattgaaggaagagaccgaatcatggcctggacagtggtgaacagtatatgtaacacaacaggggtagagaagcctaaatttcttcctgatttgtatgattacaaagagaaccggttcattgaaattggagtaacacggagggaagtccacatatattacctagagaaagccaacaaaataaaatctgagaagacacacattcacatcttttcattcactggagaggagatggccaccaaagcggactacacccttgacgaagagagcagggcaagaatcaaaactaggcttttcactataagacaagaaatggccagtaggagtctatgggattcctttcgtcagtccgaaagaggcgaagagacaattgaagaaaaatttgagattacaggaactatgcgcaagcttgccgaccaaagtctcccaccgaacttccccagccttgaaaactttagagcctatgtagatggattcgagccgaacggctgcattgagggcaagctttcccaaatgtcaaaagaagtgaacgccaaaattgaaccattcttgaggacgacaccacgccccctcagattgcctgatgggcctctttgccatcagcggtcaaagttcctgctgatggatgctctgaaattaagtattgaagacccgagtcacgagggggagggaataccactatatgatgcaatcaaatgcatgaagacattctttggctggaaagagcctaacatagtcaaaccacatgagaaaggcataaatcccaattacctcatggcttggaagcaggtgctagcagagctacaggacattgaaaatgaagagaagatcccaaggacaaagaacatgaagagaacaagccaattgaagtgggcactcggtgaaaatatggcaccagaaaaagtagactttgatgactgcaaagatgttggagaccttaaacagtatgacagtgatgagccagagcccagatctctagcaagctgggtccaaaatgaattcaataaggcatgtgaattgactgattcaagctggatagaacttgatgaaataggagaagatgttgccccgattgaacatatcgcaagcatgaggaggaactattttacagcagaagtgtcccactgcagggctactgaatacataatgaagggagtgtacataaatacggccttgctcaatgcatcctgtgcagccatggatgactttcagctgatcccaatgataagcaaatgtaggaccaaagaaggaagacggaaaacaaacctgtatgggttcattataaaaggaaggtctcatttgagaaatgatactgatgtggtgaactttgtaagtatggagttctcactcactgacccgagactggagccacacaaatgggaaaaatactgtgttcttgaaataggagacatgctcttgaggactgcgataggccaagtgtcgaggcccatgttcctatatgtgagaaccaatggaacctccaagatcaagatgaaatggggcatggaaatgaggcgctgccttcttcagtctcttcagcagattgagagcatgattgaggccgagtcttctgtcaaagagaaagacatgaccaaggaattctttgaaaacaaatcggaaacatggccaatcggagagtcacccaggggagtggaggaaggctctattgggaaagtgtgcaggaccttactggcaaaatctgtattcaacagtctatatgcgtctccacaacttgaggggttttcggctgaatctagaaaattgcttctcattgttcaggcacttagggacaacctggaacctggaaccttcgatcttggggggctatatgaagcaatcgaggagtgcctgattaatgatccctgggttttgcttaatgcatcttggttcaactccttcctcacacatgcactgaagtag

SEQ ID NO: 16 - protein, 716 aa, Influenza A virus
MEDFVRQCFNPMIVELAEKAMKEYGEDPKIETNKFAAICTHLEVCFMYSDFHFIDERGESIIVESGDPNALLKHRFEIIEGRDRIMAWTVVNSICNTTGVEKPKFLPDLYDYKENRFIEIGVTRREVHIYYLEKANKIKSEKTHIHIFSFTGEEMATKADYTLDEESRARIKTRLFTIRQEMASRSLWDSFRQSERGEETIEEKFEITGTMRKLADQSLPPNFPSLENFRAYVDGFEPNGCIEGKLSQMSKEVNAKIEPFLRTTPRPLRLPDGPLCHQRSKFLLMDALKLSIEDPSHEGEGIPLYDAIKCMKTFFGWKEPNIVKPHEKGINPNYLMAWKQVLAELQDIENEEKIPRTKNMKRTSQLKWALGENMAPEKVDFDDCKDVGDLKQYDSDEPEPRSLASWVQNEFNKACELTDSSWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAIGQVSRPMFLYVRTNGTSKIKMKWGMEMRRCLLQSLQQIESMIEAESSVKEKDMTKEFFENKSETWPIGESPRGVEEGSIGKVCRTLLAKSVFNSLYASPQLEGFSAESRKLLLIVQALRDNLEPGTFDLGGLYEAIEECLINDPWVLLNASWFNSFLTHALK

SEQ ID NO: 17 - DNA, 1497 bp, Influenza A virus
  misc_feature (1)..(1497): Synthetic cDNA construct coding for influenza A NP viral segment
atggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaagagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactgagaagcagatactgggccataaggaccaggagtggaggaaataccaatcaacaaaaggcatccgcaggccagatcagtgtgcagcctacattctcagtgcagcggaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagttga

SEQ ID NO: 18 - protein, 498 aa, Influenza A virus
MASQGTKRSYEQMETGGERQDATEIRASVGRMIGGIGRFYIQMCTELKLSDYDGRLIQNSITIERMVLSAFDERRNKYLEEHPSAGKDPKKTGGPIYRRVDGKWMRELILYDKEEIRRVWRQANNGEDATAGLTHIMIWHSNLNDATYQRTRALVRTGMDPRMCSLMQGSTLPRRSGAAGAAVKGVGTIAMELIRMIKRGINDRNFWRGENGRRTRVAYERMCNILKGKFQTAAQRAMMDQVRESRNPGNAEIEDLIFLARSALILRGSVAHKSCLPACVYGLAVASGHDFEREGYSLVGIDPFKLLQNSQVVSLMRPNENPAHKSQLVWMACHSAAFEDLRVSSFIRGKKVIPRGKLSTRGVQIASNENVETMDSNTLELRSRYWAIRTRSGGNTNQQKASAGQISVQPTFSVQRNLPFERATVMAAFSGNNEGRTSDMRTEVIRMMESAKPEDLSFQGRGVFELSDEKATNPIVPSFDMSNEGSYFFGDNAEEYDS

SEQ ID NO: 19 - DNA, 982 bp, Influenza A virus
  misc_feature (1)..(982): Synthetic cDNA construct coding for influenza A M viral segment
atgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtgatcctctcgtcattgcagcaaatatcattgggatcttgcacctgatattgtggattactgatcgtctttttttcaaatgtatttatcgtcgctttaaatacggtttgaaaagagggccttctacggaaggagtgcctgagtccatgagggaagaatatcaacaggaacagcagagtgctgtggatgttgacgatggtcattttgtcaacatagagctagagtaa

SEQ ID NO: 20 - protein, 252 aa, Influenza A virus
MSLLTEVETYVLSIIPSGPLKAEIAQRLESVFAGKNTDLEALMEWLKTRPILSPLTKGILGFVFTLTVPSERGLQRRRFVQNALNGNGDPNNMDRAVKLYKKLKREITFHGAKEVSLSYSTGALASCMGLIYNRMGTVTTEAAFGLVCATCEQIADSQHRSHRQMATTTNPLIRHENRMVLASTTAKAMEQMAGSSEQAAEAMEVANQTRQMVHAMRTIGTHPSSSAGLKDDLLENLQAYQKRMGVQMQRFK

SEQ ID NO: 21 - protein, 97 aa, Influenza A virus
MSLLTEVETPTRSEWECRCSDSSDPLVIAANIIGILHLILWITDRLFFKCIYRRFKYGLKRGPSTEGVPESMREEYQQEQQSAVDVDDGHFVNIELE

SEQ ID NO: 22 - DNA, 863 bp, Influenza A virus
  misc_feature (1)..(863): Synthetic cDNA construct coding for influenza A NS viral segment
atggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcgctacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatgaaaagtggcgagagcaattgggacagaaatttgaggaaataaggtggttaattgaagaaatgcggcacagattgaaagcgacagagaatagtttcgaacaaataacatttatgcaagccttacaactactgcttgaagtagaacaagagataagagctttctcgtttcagcttatttaatgataaaaaacacccttgtttctac

SEQ ID NO: 23 - protein, 219 aa, Influenza A virus
MDSNTMSSFQVDCFLWHIRKRFADNGLGDAPFLDRLRRDQKSLKGRGNTLGLDIETATLVGKQIVEWILKEESSETLRMTIASVPTSRYLSDMTLEEMSRDWFMLMPRQKIIGPLCVRLDQAIMEKNIVLKANFSVIFNRLETLILLRAFTEEGAIVGEISPLPSLPGHTYEDVKNAVGVLIGGLEWNGNTVRVSENIQRFAWRNCDENGRPSLPPEQK

SEQ ID NO: 24 - protein, 121 aa, Influenza A virus
MDSNTMSSFQDILMRMSKMQLGSSSEDLNGMVTRFESLKIYRDSLGETVMRMGDLHYLQSRNEKWREQLGQKFEEIRWLIEEMRHRLKATENSFEQITFMQALQLLLEVEQEIRAFSFQLI

(19) United States
(12) Patent Application Publication
(10) Pub. No.: US 2018/0326040 A1
(43) Pub. Date: Nov. 15, 2018

(54) INFLUENZA VIRUS VACCINE AND VACCINE PLATFORM

(71) Applicant: Kansas State University Research Foundation, Manhattan, KS (US)

(72) Inventors: Ben Hause, Slayton, MN (US); Emily Collin, Lenexa, KS (US)

(21) Appl. No.: 15/776,016

(22) PCT Filed: Nov. 16, 2016
(86) PCT No.: PCT/US16/62236

§ 371 Date: May 14, 2018

Related U.S. Application Data
(60) Provisional application No. 62/255,585, filed on Nov. 16, 2015.

(51) Int. Cl. A61K 39/145; C07K 14/005

(52) U.S. Cl. CPC A61K 39/145; C07K 14/005; A61K 2039/5256; C12N 2760/16322; C12N 2760/16121

(57) ABSTRACT

Attenuated live or replication-deficient chimeric swine influenza virus vaccine, and a platform to develop a multitude of other vaccines in swine and other species. The chimeric influenza virus A comprises a backbone of viral genomic segments derived from influenza A, and expresses a heterologous surface protein (influenza D hemagglutinin esterase fusion protein), and optionally at least one other heterologous protein, such as an antigenic component of a target pathogen.

### CROSS-REFERENCE TO RELATED APPLICATIONS

The present application claims the priority benefit of U.S. Provisional Patent Application Ser. No. 62/255,585, filed Nov. 16, 2015, entitled Swine Influenza Virus Vaccine and Vaccine Platform, incorporated by reference in its entirety herein.

### SEQUENCE LISTING

The following application contains a sequence listing in computer readable format (CRF), submitted as a text file in ASCII format entitled “SequenceListing,” created on Nov. 15, 2016, as 68 KB. The content of the CRF is hereby incorporated by reference.

### BACKGROUND OF THE INVENTION

### Field of the Invention

The present invention relates to chimeric, attenuated live or replication-deficient swine influenza virus vaccine.

### Description of Related Art

Swine influenza virus (SIV) and porcine epidemic diarrhea virus (PEDV) are two of the most economically important viruses to the U.S. swine industry. While commercial vaccines are available to aid in disease prevention, their efficacy is lacking. A significant shortcoming with these vaccines is that they are composed of inactivated virus and are administered parentally. This leads to a systemic humoral response dominated by IgG. SIV and PEDV infect pigs via mucosal surfaces where protection is best mediated by secretory IgA, an antibody isotype poorly induced by parental vaccination. Next generation vaccines that stimulated mucosal immunity are needed to improve SIV and PEDV control strategies.

Despite a long history of both inactivated commercial and autogenous vaccine use for SIV, the economic impact of SIV continues to increase. While inactivated vaccines with antigens well matched to challenge virus induce protective immunity, the ever-increasing genetic diversity of influenza virus prohibits coverage of emerging and variant viruses. Inactivated vaccine formulations are updated periodically however are unable to keep pace with the changing nature of influenza viruses. Newer vaccine technologies that induce broad, cross reactive immunity to conserved viral antigens are needed to control influenza.

The segmented genome of influenza A consists of 8 genomic segments (See FIG. 1) of linear, negative polarity, single-stranded RNAs which encode for polypeptides: RNA polymerase subunits PB1, PB2, and PA and nucleoprotein (NP) which form the nucleocapsid; the matrix proteins (M1, M2); the nonstructural protein (NS1, NS2); and two surface glycoproteins which project from the lipoprotein envelope: hemagglutinin (HA) and neuraminidase (NA). Segments 4 and 6 encode HA and NA genes, respectively. Influenza A virions bind to cellular receptors via their HA proteins and utilize the NA protein to release virus from receptors.

IDV is an RNA virus consisting of 7 genomic segments (See FIG. 1). IDV was recently discovered and is common in cattle (Hause et al. 2013. Isolation of a novel swine influenza virus from Oklahoma in 2011 which is distantly related to human influenza C viruses. PLoS Pathogens 9(2):e1003176. doi: 10.1371/journal.ppat.1003176; Hause et al. 2014. Characterization of a novel influenza virus in cattle and Swine: proposal for a new genus in the Orthomyxoviridae family. MBio 5(2):e00031-14. doi: 10.1128/mBio.00031-14). One significant difference between IAV and IDV is that IDV uses a hemagglutinin esterase fusion (HEF) protein for receptor binding and release functionality as opposed the HA/NA utilized by IAV. In this way, IDV is more related to human influenza C virus (ICV).

### SUMMARY OF THE INVENTION

The present invention is broadly concerned with an attenuated live or replication-deficient swine influenza virus; a platform to develop a multitude of vaccines (PCV2, PEDV, PDCoV, BCV); and a platform that may work in other species to treat influenza (e.g., equine, bovine, human, canine, feline, etc.). The chimeric influenza A virus comprises a backbone of viral genomic segments derived from influenza A, and expresses a heterologous surface protein, i.e., influenza D hemagglutinin esterase fusion (HEF) protein. The chimeric virus serves as a vector for delivering conserved viral proteins (influenza A) to an animal in a live virus format to induce robust protective immunity, while limiting viral replication and the incidence of clinical disease. Elastase-dependent versions of the virus can be prepared to further enhance the avirulence of the vaccine vector.

Also disclosed herein are vaccine compositions useful for inducing an immune response against viral infection. The vaccine compositions comprise a therapeutically-effective amount of chimeric influenza A virus dispersed in a pharmaceutically-acceptable carrier; and optionally, one or more adjuvants, active agents, preservatives, buffering agents, or salts dispersed in the carrier. Use of live vaccine facilitates the creation of broadly cross reactive protective immune responses to influenza.

Advantageously, the vaccine platform can be further modified for heterologous gene expression to create multi-valent vaccines. For example, chimeric viruses carrying genes/expressing proteins including Porcine Reproductive and Respiratory Syndrome virus (PRRS or PRRSV) nucleocapsid, Porcine epidemic diarrhea virus (PEDV) spike protein, porcine circovirus (PCV) capsid protein and the reporter protein green fluorescent protein from jellyfish can be created, among others. In addition to inducing a protective immunity to influenza, such vaccines would induce a protective immunity to other pathogens, depending upon the chimeric platform used. In addition, the vaccines are designed for mucosal (intranasal) delivery, which elicits a more robust immune response to the vaccine.

Also described herein are methods of vaccinating a subject to induce an immune response against viral infection. The methods comprise administering a vaccine composition according to the embodiments described herein to the subject. Kits for vaccination are also disclosed, which comprise a vaccine composition according to the embodiments described herein, and instructions for administering the vaccine composition to the subject.

This disclosure is also concerned with use of a composition according to the embodiments described herein for vaccination against viral infection or to prevent or mitigate viral infection in a subject.

Also described herein are kits for study and/or generation of chimeric influenza A virus strains. The kits comprise vectors encoding for backbone viral segments derived from influenza A to produce a platform virus; chimeric vectors encoding for HA and NA packaging constructs in which heterologous polynucleotide sequences encoding for open reading frames for one or more heterologous proteins can be inserted; and instructions for transfecting cells with the vectors to generate the chimeric virus strains.

A synthetic cDNA is also described herein which encodes for HEF surface protein useful for generating chimeric influenza A viruses comprising SEQ ID NO:4.

Synthetic cDNA vectors useful for generating chimeric influenza A viruses are also disclosed. The cDNA chimeras comprise noncoding regions and viral packaging sequences derived from a native HA polynucleotide segment of influenza A, and a heterologous polynucleotide sequence encoding for an open reading frame for one or more heterologous proteins. In some embodiments, the cDNA comprises SEQ ID NO:2 or SEQ ID NO:3, where n indicates the location where the heterologous polynucleotide sequence is inserted.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration of the viral segments and surface proteins of Influenza A, Influenza D, and the inventive Influenza A/D chimera virus;

FIG. 2 shows images of swine testicle cells infected with chimera IAV/IDV virus expressing PEDV S1 as visualized by (A) indirect IFA using PEDV S1 monoclonal antibody; or (B) rabbit polyclonal antiserum generated against PEDV S1.

FIG. 3 are images of swine testicle cells infected with chimera IAV/IDV virus expressing PCV2 capsid as visualized by direct IFA using FITC-conjugated polyclonal antisera generated against PCV2;

FIG. 4 is a graph of the elastase-dependent in vitro growth kinetics for the mutated chimera virus, showing the average TCID₅₀ time-points of D/OK-pN1, D/OKelast-pN1 without elastase supplementation, and D/OKelast-pN1 with elastase supplementation;

FIG. 5 is a graph of the viral Shedding from nasal swabs post-challenge in swine based upon the average qRT-PCR CTs for each group at day 0, 1, 3, and 5 post-challenge; and

FIG. 6 shows the data of BALF and lung TCID₅₀ in correlation to IgA ELISA, where the left vertical axis indicates the average OD of BALF samples on IgA ELISA, the right vertical axis is the TCID₅₀ /mL of influenza in BALF and lungs. [symbol] Indicates group A lung and BALF titers were significantly lower (0) at day 5 compared to day 3 and group A IgA was significantly higher at day 5 than day 3.

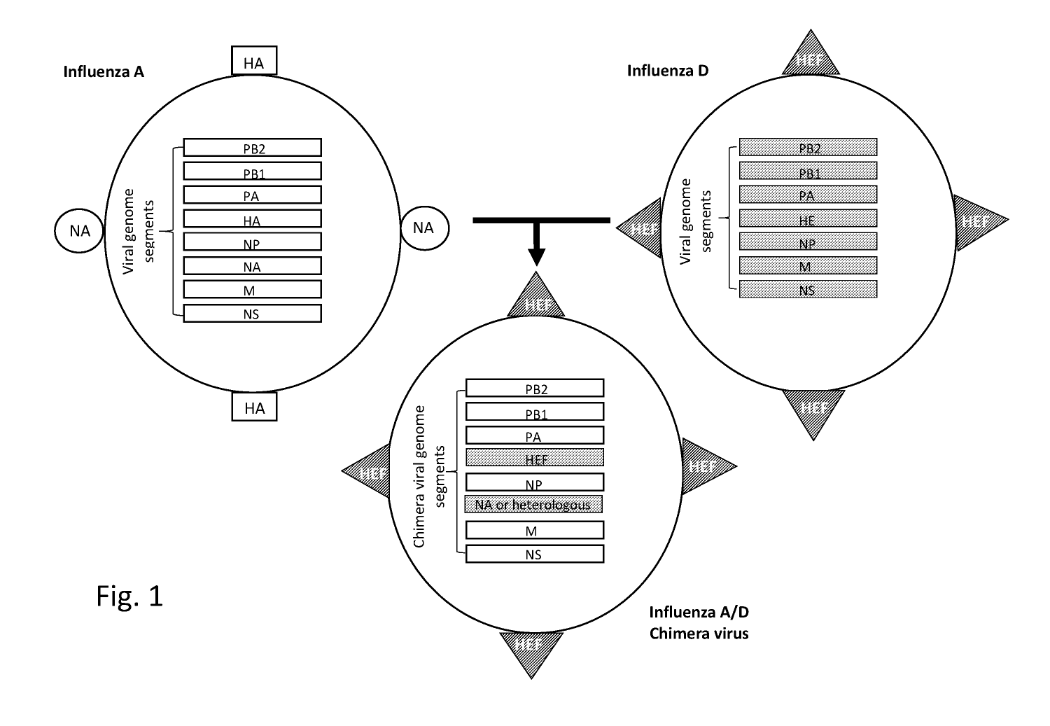

### DETAILED DESCRIPTION

The present invention is concerned with attenuated live or replication-deficient influenza virus vaccines based upon a chimeric virus platform for use in treatment or prevention of swine influenza virus and other diseases. Broadly, the virus platform comprises a chimeric influenza A virus where the native hemagglutinin (HA) and/or neuraminidase (NA) genes are replaced with a gene from another pathogen. As such, the chimeric virus expresses at least one heterologous protein. More specifically, the chimeric virus expresses a surface glycoprotein from influenza D, hemagglutinin esterase fusion protein (HEF). The chimeric virus can be further engineered to express additional heterologous sequences. These chimeric viruses are avirulent in swine and when administered intranasally induce a broadly cross reactive protective immune response to SIV.

The viruses described herein are referred to as being “modified” or “chimeric,” which means that they differ from wild-type virus strains and have non-wild type genomes and virions in which at least a portion thereof originates from a different species or strain. More specifically, the chimeric (non-wild type) influenza viruses incorporate backbone genome segments of influenza A, heterologous surface protein HEF of influenza D, and optionally a heterologous antigenic component from a target pathogen. These chimeric viruses are “synthetic” in that the individual viral segments are synthetically constructed from oligonucleotides to form functional viral segments, as discussed in more detail below, and then the segments are expressed to generate live, replicating virus particles with heterologous surface proteins. These live, synthetic viruses are useful in vaccine formulations for eliciting immune responses against the target pathogen. In general, the “target” pathogen refers to the strain or strains against which the vaccine is designed to have prophylactic (or therapeutic) effect and provide immunoprotection to the vaccinated subject.

The native influenza A virus genome consists of eight segments of negative-sense single-stranded RNA, encoding six internal proteins (PB2, PB1, PA, NP, M, and NS), and the two surface glycoproteins (HA and NA). The viral particle comprises a lipid bilayer envelope, within which all eight RNA genomic segments reside, and the outer layer of the lipid envelope is spiked with multiple glycoproteins HA and NA (and a small number of M2). During replication, the genomic viral RNA is transcribed into positive-strand mRNA and negative-strand genomic cRNA in the nucleus of the host cell. Each of the eight genomic segments is packaged into ribonucleoprotein complexes that contain, in addition to the RNA, NP and a polymerase complex (PB1, PB2, and PA). The native influenza A virus is characterized by a viral particle (virion) having surface HA and NA extending from the particle envelope, and viral RNAs that make up the eight-segment genome inside the particle core and bound to ribonuclear proteins (RNPs). The chimeric virus is characterized by heterologous surface proteins in place of HA and/or NA, and optionally at least one additional heterologous sequence in the genome segment.

The viral segments in the chimeric virus are derived from sequence information for known virus strains. As used herein the term “derived from” means that the modified or chimeric virus is synthetically generated from genome segments constructed directly using the known (cDNA) sequences for the corresponding segment of each strain and chemical or enzymatic synthesis and assembly of the oligonucleotides. For example, internal segment for PB2 of the synthetic virus is “derived from” PB2 of influenza A, in that the genome segment encoding for PB2 of the synthetic virus is constructed synthetically using the known coding sequence for PB2 of influenza A as the template. Various synthetic genetics techniques are known in the art. Briefly, the synthetic constructs comprise coding sequences (cDNA) for expressing one or more viral RNA segment(s) of an influenza virus genome. The encoded segments can be expressed and then function as viral RNAs which can be packaged into virions. For example, the synthetic expression construct can drive expression in a eukaryotic cell of viral segments encoded therein. The expressed viral segment RNA can be translated into a viral protein that can be incorporated into a virion. The synthetic expression construct can encode all (eight) viral segments necessary to produce an influenza virus, or the viral segments can be provided in multiple expression construct(s).

In one or more embodiments, each of the eight viral segments is synthesized into individual expression constructs, each containing one copy of the viral cDNA. For example, Hoffmann et al. disclose an eight-plasmid DNA transfection system for the rescue of infectious influenza A virus from cloned cDNA (Proc. Natl. Acad. Sci., vol. 97, no. 11, A DNA transfection system for generation of influenza A virus from eight plasmids (2000), see also U.S. Pat. No. 6,951,754, incorporated by reference herein). In this plasmid-based expression system, synthesized cDNA for the virus is inserted between the RNA polymerase I (pol I) promoter and terminator sequences. The plasmids are transfected into a eukaryotic cell system. This entire pol I transcription unit is flanked by an RNA polymerase II (pol II) promoter and a polyadenylation site. The orientation of the two transcription units allows the synthesis of negative-sense viral RNA and positive-sense mRNA from one viral cDNA template. The mRNAs are translated into viral proteins. Interaction of these molecules derived from the cellular transcription and translation machinery results in the interaction of all synthesized molecules (vRNPs and structural proteins) to generate functional (infectious) viral particles. Thus, the viral genomic RNA is the reverse complement of the cDNA sequences disclosed.

In the chimeric virus, at least one of the native HA or NA segments of influenza A is replaced by a construct encoding for a heterologous gene product. More particularly, HA or NA packaging constructs are synthesized in which the open reading frame for the heterologous gene product is combined with noncoding regions and viral packaging sequences derived from influenza A, as described in more detail below. Preferably, the backbone viral genome segments are synthesized according to backbone sequences of an influenza A strain. As used herein, reference to “backbone” or “platform” sequences refers to sets of genome segments encoding influenza virus proteins other than surface proteins HA, NA (and thus, generally refers to the internal core genome segments). Thus, the “platform” strain or virus refers to the virus from which backbone segments (or sequence information relating thereto) originate. More specifically, the six internal protein coding vRNAs for PB2, PB1, PA, NP, M, and NS from influenza A can be synthesized (via cDNA) from known or determined sequences to produce viral backbone genome segments. Sequence fragments may also be used so long as they are “functional fragments” meaning that they nonetheless encode a functional protein for the virus from which the sequence was derived.

In one or more embodiments, the backbone segments are synthesized from synthetic constructs of influenza A selected from the group consisting of SEQ ID NO:11 (PB1), SEQ ID NO:13 (PB2), SEQ ID NO:15 (PA), SEQ ID NO:17 (NP), SEQ ID NO:19 (M), and SEQ ID NO:22 (NS). In one or more embodiments, the backbone segments encode for one or more viral proteins selected from the group consisting of SEQ ID NO:12 (PB1), SEQ ID NO:14 (PB2), SEQ ID NO:16 (PA), SEQ ID NO:18 (NP), SEQ ID NO:20 (M1), SEQ ID NO:21 (M2), SEQ ID NO:23 (NS1), and SEQ ID NO:24 (NS2).

Heterologous gene(s) or genome segment(s) encoding a complete open reading frame for the heterologous protein to replace native HA and/or NA are likewise synthesized based upon sequence information for the target pathogen to be vaccinated against. In one or more embodiments, native HA is replaced with a synthetic construct encoding for HEF from influenza D. In one or more embodiments, native NA is replaced with a synthetic construct encoding for HEF from influenza D. In one or more embodiments, native HA is replaced with a synthetic construct encoding for a heterologous antigenic component of a non-influenza pathogen. In one or more embodiments, native NA is replaced with a synthetic construct encoding for a heterologous antigenic component of a non-influenza pathogen. Preferably, at least one of NA or HA is replaced with a synthetic construct encoding for HEF from influenza D, which serves to attenuate the influenza A chimera when constructed. In optional embodiments, the other segment (NA or HA, as applicable) is synthesized from synthetic constructs of native NA or HA of influenza A, and as such, the chimeric virus expresses HEF in combination with either native NA or HA, as applicable. In some embodiments, both NA and HA are replaced with heterologous segments encoding for heterologous proteins, where at least one segment encodes for heterologous HEF and the other segment encodes for a heterologous antigenic component of a non-influenza pathogen. As such, the chimeric influenza A/D virus does not express either native NA (e.g., SEQ ID NO:6) or HA. An advantage of the chimera virus is that the lack of HA in the virus prevents maternal antibody interference in swine, as pigs are largely seronegative to IDV. This will allow use of the vaccine in young (<3 weeks) pigs, unlike existing vaccines.

The sequence of the heterologous coding region in either segment HA or NA can be synthesized (via cDNA) from known or determined sequences from influenza D and/or the target pathogen. It is preferred that the complete coding region is used, although fragments may be used so long as they encode a functional (immunogenic/antigenic) HEF protein and/or antigenic component of the virus from which the coding region is derived (i.e., are “functional fragments”). The heterologous cDNA is synthetized and flanked by control sequences, and more particularly, is a chimeric gene in which the noncoding regions and viral packaging sequences from the platform influenza virus (influenza A) is retained. That is, in the synthesized heterologous gene sequence, the protein open reading frame of the platform influenza HA or NA sequence is replaced by the protein open reading frame sequence for HEF or the antigenic components of the target pathogen. If necessary, silent substitutions or other mutations can be introduced to disrupt the native packaging signals in the heterologous sequence terminal coding regions. Thus, the HA and NA viral genome segments are each chimeric genes comprising (consisting essentially, or even consisting of) influenza A viral packaging sequence(s), influenza A non-coding regions, and a heterologous sequence open reading frame encoding for HEF or a non-influenza antigenic component. An exemplary HA packaging construct is in SEQ ID NO:2, where n in such sequence indicates the location where the HEF protein or antigenic component coding cDNA is inserted into the influenza A platform HA sequence. In one or more embodiments, the HA segment is a synthetic construct encoding for HEF comprising (consisting essentially of or even consisting of) SEQ ID NO:4. In one or more embodiments, the HA segment encodes for a protein having SEQ ID NO:l.

An exemplary NA packaging construct is in SEQ ID NO:3), where n in such sequence indicates the location where the HEF protein or antigenic component coding cDNA is inserted into the influenza A platform NA sequence.

An added benefit of the chimeric virus is that it can be used as a vector to produce heterologous antigens, allowing for bivalent vaccines from a single vector. That is, another feature of the chimeric influenza A/D viruses is consolidation of receptor binding and destruction activities to a single protein, HEF. This leaves vacant a genomic RNA segment (either HA or NA) that can be used for heterologous gene expression. Importantly, the added benefit of this vaccine design is that protection can be afforded for a second antigen beyond influenza. This is extremely important as labor to vaccinate animals is a significant factor in vaccine usage. The convenience of a single vaccination that provides superior protection to two diseases is highly advantageous.

Thus, in some embodiments, the NA packaging construct encodes for a heterologous antigenic component of a target pathogen. As used herein, “antigenic component” refers to the entire antigen or portion/fragment of an antigen capable of eliciting an immune response in a subject against the pathogen from which that antigen derives. Constructs can be synthesized for expressing antigenic components of a pathogen such as Porcine reproductive and respiratory syndrome virus (PRRS or PRRSV), Porcine epidemic diarrhea virus (PEDV), Porcine Circovirus Type 2 (PCV2), Porcine deltacoronavirus (PDCoV), Bovine coronavirus (BCV), Bovine respiratory syncytial virus (BRSV), and the like. In one or more embodiments, the NA segment is selected from the group consisting of SEQ ID NO:7 (PRRS), SEQ ID NO:8 (PEDV), and SEQ ID NO:9 (PCV2). Thus, after packaging and rescue of the chimeric virus, the resulting viral RNA encodes a polypeptide that generally preferably comprises at least an epitope from the target pathogen, such that it will elicit an immune response when it is introduced into a subject, or when it is synthesized within the cells of a host or a subject (in vitro or in vivo). For example, one could clone various permutations of the PEDV spike (S) gene into the NA (or HA) segment to generate an influenza virus that expresses PEDV spike antigens. In one or more embodiments, the NA segment may encode for a spike subunit, nucelocapsid, or other antigenic protein fragment of the target pathogen.

As noted above, the HA segment may instead be used to express the heterologous antigenic component (while the NA segment is used to express HEF). In some embodiments, the NA or HA packaging construct may also encode for a marker protein (e.g., GFP), in lieu of an antigen component, such as illustrated in SEQ ID NO:10.

Thus, it will be appreciated that when a target pathogen is identified, the HA and NA segments of that strain may be synthesized into cDNA and included in an expression construct along with the influenza A backbone segments (in the same or a different construct) for generating the chimeric viral particles. For example, the synthesized segments can be inserted or cloned into suitable vectors for propagation, and preferably plasmid vectors. In one or more embodiments, individual plasmids can be generated for each of the eight influenza segments, one encoding for each of PB2, PB1, PA, NP, M, and NS, as well as chimeric HA and NA. The plasmids are then transfected into appropriate cells for generation of the chimeric virions. The resulting cell transfection supernatants can then be incubated with various cells and/or embryonated chicken eggs and passaged followed by propagation. Suitable cells are preferably mammalian, although avian or insect cells can also be used.

In one or more embodiments, the chimera viruses are further modified such that the HEF trypsin cleavage site is mutated to that of elastase. The modification of the trypsin cleavage site to elastase attenuates the virus in vivo but still allows for virus growth in vitro through elastase supplementation of the cell culture media. IAV/IDV chimera viruses with elastase-dependent cleavage will not replicate in swine. The viruses will infect swine cells in vivo however will only undergo a single abortive replication cycle. Infectious virus will not be shed into the environment.

Regardless, the resulting chimeric virus can be used for live attenuated or replication-deficient vaccines against influenza type A to prevent or mitigate influenza infection in the subject. As used herein, the term “vaccine” refers to an immunogenic composition capable of eliciting partial or complete immunogenic protection against a disease or condition in the subject to which it has been administered. Although vaccines are generally considered prophylactic, the vaccines may be used for therapeutic treatment of a disease or a condition. Compositions according to the embodiments disclosed herein are useful in treating viral infection from influenza and other pathogens in a subject (e.g., swine) and/or preventing or reducing clinical symptoms of infection. Such clinical symptoms vary from disease to disease. Thus, embodiments described herein have therapeutic and/or prophylactic uses, and in particular can be used for prophylactic treatment of a viral infection. In general, the compositions are administered prophylactically, that is, before the subject demonstrates detectable clinical signs of an infection, such that the subject develops an adaptive immune response to infection by the virus. As such, the methods are useful for preventing the development of observable clinical symptoms from viral infection, and/or reducing the incidence or severity of clinical symptoms, and/or effects of the infection, and/or reducing the duration of the infection/symptoms/effects, and/or reducing the amount and/or duration viral shedding/viremia (e.g., excretion or expulsion of the virus or viral particles from an infected subject), as compared with unvaccinated control animals. Thus, the composition may only partially prevent and/or lessen the extent of morbidity due to the viral infection (i.e., reduce the severity of the symptoms and/or effects of the infection, and/or reduce the duration of the infection/symptoms/effects), as compared with unvaccinated control animals. Yet, the composition is still considered is still considered to treat or “prevent” the target infection or disease, even though it is not 100% effective.

The vaccines comprise the chimeric influenza A/D virus described herein dispersed in a pharmaceutically-acceptable carrier. The term carrier is used herein to refer to diluents, excipients, vehicles, and the like, in which the chimeric influenza A/D virus may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would naturally be selected to minimize any degradation of the chimeric influenza A/D virus or other agents and to minimize any adverse side effects in the subject, as would be well known to one of skill in the art. Pharmaceutically-acceptable ingredients include those acceptable for veterinary use as well as human pharmaceutical use, and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), aqueous dextrose solutions, aqueous glycerol solutions, ethanol, normal allantoic fluid, various oil-in-water or water-in-oil emulsions, as well as dimethyl sulfoxide (DMSO) or other acceptable vehicles, and the like.

The vaccine can comprise a therapeutically effective amount of chimeric influenza A/D virus dispersed in the carrier. As used herein, a “therapeutically effective” amount refers to the amount that will elicit the biological or medical response of a tissue, system, or subject that is being sought by a researcher or clinician, and in particular elicit some desired protective effect as against the viral infection by priming or stimulating an immune response specific for one or more strains of influenza virus (and preferably at least the target strain). One of skill in the art recognizes that an amount may be considered therapeutically “effective” even if the condition is not totally eradicated or prevented, but it or its symptoms and/or effects are improved or alleviated partially in the subject. In some embodiments, the composition will comprise from about 5% to about 95% by weight of chimeric influenza A/D virus described herein, and preferably from about 30% to about 90% by weight of the chimeric influenza A/D virus, based upon the total weight of the composition taken as 100% by weight. In some embodiments, combinations of more than one type of the described chimeric influenza A/D virus can be included in the composition, in which case the total levels of all such viral particles will preferably fall within the ranges described above. Such multi-valent vaccines may be preferred for use in vaccination against the flu virus.

Other ingredients may be included in the composition, such as adjuvants, other active agents, preservatives, buffering agents, salts, other pharmaceutically-acceptable ingredients, including residual amounts of ingredients used in vaccine manufacturing. The term “adjuvant” is used herein to refer to substances that have immunopotentiating effects and are added to or co-formulated in the vaccine composition in order to enhance, elicit, and/or modulate the innate, humoral, and/or cell-mediated immune response against the vaccine components. Suitable adjuvants include: aluminum salts, such as aluminum hydroxide, aluminum phosphate, alum (potassium aluminum sulfate), or mixed aluminum salts, peptides, oil or hydrocarbon emulsions, or any other adjuvant deemed suitable for human or animal use. In some embodiments, the vaccine is substantially free of any adjuvants, where the term “substantially free” means that the ingredient is not intentionally added or part of the composition, although it is recognized that residual or incidental amounts or impurities may be present in low amounts (e.g., less than about 0.1% by weight and preferably less than about 0.01% by weight, based upon the total weight of the composite taken as 100% by weight). Other active agents that could be included in the composition include other antiviral compounds or any immunogenic active components (e.g., antigens) such as those that resemble a disease-causing microorganism or infectious agent, and/or are made from weakened or killed forms of the same, its toxins, subunits, particles, and/or one of its surface proteins, such that it provokes an immune response to that microorganism or infectious agent. In addition to live, modified, or attenuated vaccine components, active agents using synthetic peptides, carbohydrates, or antigens can also be used. Antibiotics can also be used as part of vaccine production and may be present in small amounts in the vaccine, such as neomycin, polymyxin B, streptomycin and gentamicin. In some embodiments, the vaccine composition is substantially free of any other active (immunogenic) agents, other than the chimeric influenza A/D virus and optional adjuvant, dispersed in the carrier.

In use, the vaccine composition is administered to a subject. Various routes of administration can be used depending upon the particular carrier and other ingredients used. For example, the vaccine can be injected intramuscularly, subcutaneously, intradermally, or intravenously using a needle and syringe, or a needleless injection device. The vaccine can also be administered mucosally, such as intranasal administration. In some embodiments, mucosal administration is particularly preferred. For intranasal administration, the vaccine composition is usually administered through the nasal passage as drops, large particle aerosol (greater than about 10 microns), or spray into the upper respiratory tract. While stimulation of a protective immune response with a single dose is preferred, additional dosages can be administered, by the same or different route, to achieve the desired prophylactic effect. The vaccine can also be administered using a prime and boost regime if deemed necessary. In some embodiments, the methods described herein are useful for eliciting an immune response against influenza infection, as described above.

Such an “immune response” includes, for example, the production or activation of antibodies, B cells and/or the various T cells, directed specifically to an antigen or antigenic component of the influenza or other target pathogen. The immune response will be demonstrated by a lack of observable clinical symptoms, or reduction of clinical symptoms normally displayed by an infected animal, faster recovery times from infection, reduced duration or amount of viral shedding, and the like. Accordingly, vaccinated animals will display resistance to new infection (or observable signs of infection) or reduced severity of infection, as compared to unvaccinated animals. “Reducing” the incidence, severity, and/or duration of clinical symptoms and/or viral shedding, means reducing the number of infected animals in a group, reducing or eliminating the number of animals exhibiting clinical signs of infection, or reducing the severity of any clinical signs that are present in the animals, in comparison to wild-type infection in unvaccinated animals.

In some embodiments, the vaccine can be provided in unit dosage form in a suitable container. The term “unit dosage form” refers to a physically discrete unit suitable as a unitary dosage for human or animal use. Each unit dosage form may contain a predetermined amount of the vaccine (and/or other active agents) in the carrier calculated to produce the desired effect. In other embodiments, the vaccine can be provided separate from the carrier (e.g., in its own vial, ampule, sachet, or other suitable container) for on-site mixing before administration to a subject. A kit comprising the vaccine is also disclosed herein. The kit further comprises instructions for administering the vaccine to a subject. The virus can be provided as part of a dosage unit, already dispersed in a pharmaceutically-acceptable carrier, or it can be provided separately from the carrier. The kit can further comprise instructions for preparing the virus for administration to a subject, including for example, instructions for dispersing the virus in a suitable carrier.

Using the methodology and technology described herein, different subtype attenuated influenza vaccines can be developed and used for swine and other species including, but not limited to, human, canine, equine, feline, avian, bovine, primate, rodents, and the like. For example, the chimeric virus could be used as a chimeric influenza D virus vaccine in cattle. Data from the pig study below showed a robust humoral antibody response (hemagglutination inhibition assay) in vaccinated pigs to IDV. While IDV is not as serious of a concern to pigs, there is growing consensus that IDV is part of the bovine respiratory disease complex. The pig results suggests that the chimera vaccine could be used in cattle as a vaccine to protect against IDV as well as a vector to protect against other pathogens.

The methods can be also applied for clinical research and/or study. Thus, kits for study and/or generation of additional chimeric virus strains are also described herein. The kits comprise vectors (plasmids) as described herein encoding for the influenza A backbone genome segments to produce the platform virus. The kit can also include vectors for HA and NA packaging constructs. The kit may include plasmids for subsequently inserting the determined sequences into the HA and NA constructs for generation of the chimeric virus. The kit may further include additional components, including cells, culture medium, buffers, along with instructions for their use to generate the chimeric viruses.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Introduction

In this work, we have created chimeric IAV/IDV viruses as a platform technology to express heterologous antigens. The backbone of the virus is pandemic H1N1 containing native genes PB2, PB1, PA, NP, M, and NS. The pH1N1 HA gene in the HA segment has been replaced with the HEF gene of IDV (the HEF gene from strain D/swine/Oklahoma/1334/2011, see U.S. Pub No. 2013/0195915, incorporated by reference herein). Chimera viruses that utilize IDV HEF for receptor binding and release have been rescued. The empty genomic segment once occupied by NA has been used to express heterologous genes using a NA packaging construct. Genes cloned into the NA segment include the marker green fluorescent protein (GFP), porcine reproductive and respiratory syndrome virus nucleocapsid (PRRS N), porcine epidemic diarrhea virus spike subunit (PEDV S1), porcine deltacoronavirus spike subunit (PDCoV S1), porcine circovirus capsid (PCV2 cap), and bovine coronavirus spike subunit (BCV S1)

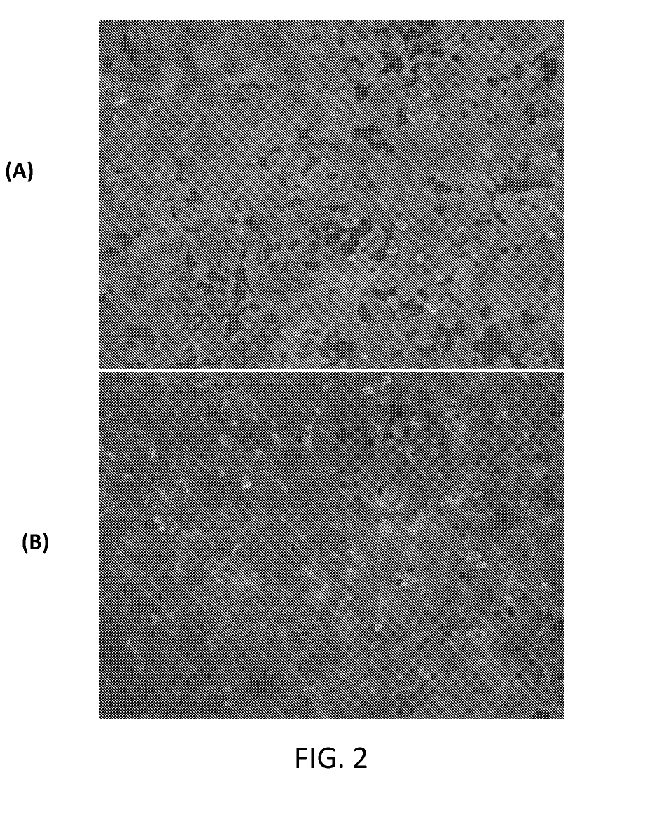

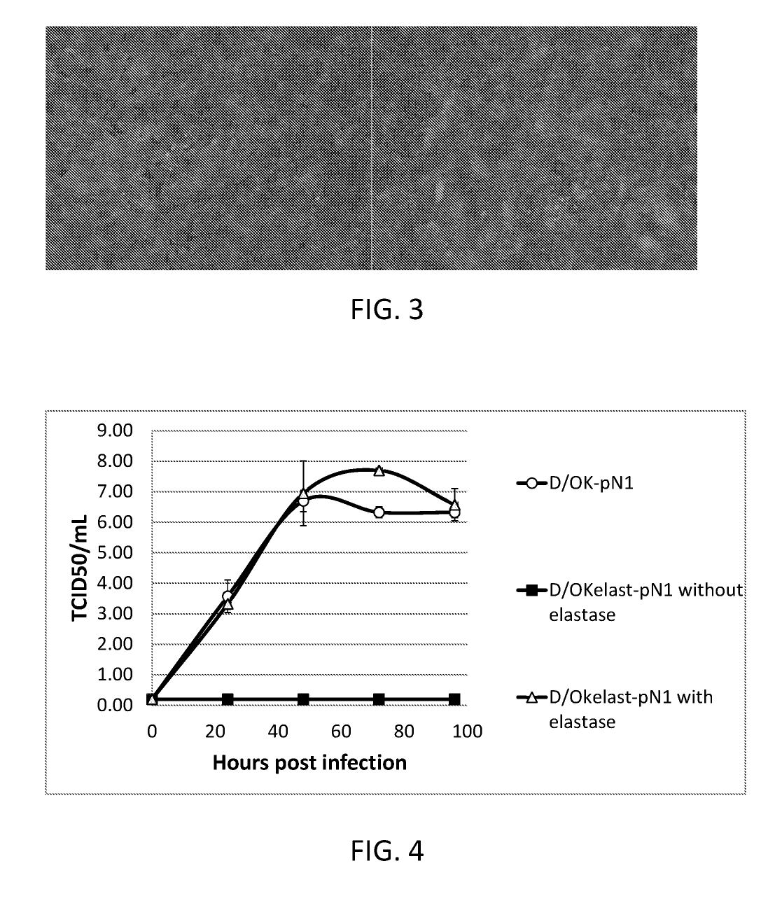

Virus bearing GFP fluoresces when visualized using a fluorescent microscope, proving that GFP is expressed and properly folded. Likewise, direct immunofluorescent detection of the chimera virus expression PRRS N using monoclonal antibody SDOW17 indicated successful N protein expression. PEDV S1 expression was verified by indirect IFA using both a monoclonal antibody and polyclonal antiserum directed against S1 (FIG. 2). PCV2 capsid expression was verified by direct IFA using polyclonal antiserum (FIG. 3).

We were able to rescue all chimera viruses based on cytopathic effects in cells and all viruses grew to high titers (>640 HA units/mL) in cell culture based on standard hemagglutination assays.

As the vaccine lacks the highly variable, immunodominant HA protein, immunity will be focused on conserved viral proteins making it effective against a broad range of influenza viruses and not subject to maternal antibody interference, enabling vaccination of young pigs. The vaccines can also be designed to be given intranasally or orally to sows prior to farrowing, thus stimulating IgA secreting immunocytes that migrate to the mammary glands where they secrete IgA to the colostrum and milk. The intranasal route of administration will also elicit superior immunity for PEDV as compared to conventional vaccines, which when administered to sows will provide lactogenic immunity to pigs. Construction and evaluation of both attenuated live and single cycle infectious virus candidate vaccines assures the highest combination of safety and efficacy.

### Example 1

An influenza A virus reverse genetic system developed for the pandemic H1N1 virus A/California/04/2009(H1N1) was used as the backbone for chimera virus construction. This reverse genetic system consists of eight plasmids derived from the plasmid pHW2000, a bi-directional expression plasmid that transcribes messenger RNA cloned into the cloning site of the plasmids in both orientations. All eight influenza genomic segments are individually cloned into pHW2000 and co-transfection of the plasmids into 293T/MDCK cell culture results in generation of infectious virus.

To generate chimera IAV/IDV viruses, the HEF gene of D/OK (residues 86-2080 of SEQ ID NO:4) was cloned into pHW2000 along with the HA packaging sequences of IAV (residues 1-85 and 2081-2214 of SEQ ID NO:4). To accomplish this, the previously identified packaging sequences of IAV HA, consisting of the 3′ and 5′ non coding regions plus the 45 nucleotides proximal to the start codon and the 80 nucleotides proximal to the stop codon of the HA ORF, were synthesized (Gao et al. 2008. A seven-segmented influenza A virus expression the influenza C virus glycoprotein HEF. J. Virol 82:6419-26). The synthetic construct also included NotI and SbfI restriction sites connected by a short linker sequence to enable subsequent cloning of the HEF ORF. Additionally, the start codon of the native HA gene contained in the synthetic construct was mutated in silico to CTG to prevent transcription from the native HA start site. The synthetic DNA fragment containing the HA packaging sequences was amplified using HA segment specific primers described by Hoffman et al. (A DNA transfection system for generation of influenza A virus from eight plasmids. Proc. Natl. Acad. Sci. 97:6108-13, 2000) and digested with restriction enzyme BsmBI and ligated into a similarly digested pHW2000. The resulting plasmid (pHA_{packaging}) contains HA packaging sequences with NotI/SbfI restriction sites for cloning and expressing genes in the influenza HA segment (SEQ ID NO:4). Primers were next designed to amplify the HEF gene of D/OK and included 5′-NotI and 3′-SbfI restriction sites. Following amplification of D/OK HEF ORF, the amplicon was digested with NotI and SbfI and ligated into a similarly digested pHA_{packaging}. Correct clones were verified by restriction digest mapping and DNA sequencing. The resulting plasmid, pD/OK HEF, contains the complete ORF of D/OK HEF flanked by IAV HA packaging sequences (SEQ ID NO:4).

### Example 2

Rescue of recombinant viruses was performed as previously described (Hoffmann et al. 2000). In brief, 293T and MDCK cells were co-cultured in Opti-MEM I containing 5% FBS in 6-well plates approximately 1×10⁶ cells of each 293T and MDCK approximately 18 hours prior to transfection. One hundred nanograms of each of the eight plasmids were pooled in 100 μL of Opti-MEM I and combined with 100 μL Opti-MEM containing 3 μL Lipofectamine (Invitrogen) and incubated at room temperature 15 minutes before being diluted to 1 mL with Opti-MEM I and transferred to a single well of the 6-well plate. Plates were incubated at 37° C. with 5% CO₂ for 6 hours before the transfection mixture was replaced with Opti-MEM I. At 24 hours post transfection, 1.5 mL was transferred to a 6-well plate of confluent MDCK cells and 1.5 mL of DMEM containing 1 μg/mL of TPCK-treated trypsin was added. Viruses were harvested on day 5 post infection and their titers determined by the HA assay. The complete genomes of rescued viruses were determined to verify their authenticity.

### Example 3

Using the plasmids described in Example 1 and the methods described in Example 2, an eight segmented IAV was constructed where the pH1N1 HA gene was replaced with that of the D/OK HEF gene. Plasmids derived from pHW2000 containing IAV pH1N1 PB2 (SEQ ID NO:13), PB1 (SEQ ID NO:11), PA (SEQ ID NO:15), NP (SEQ ID NO:17), NA (SEQ ID NO:5), M (SEQ ID NO:19), and NS (SEQ ID NO:22) genomic segments were co-transfected into a co-culture of 293T/MDCK cells along with pD/OK HEF. Viral replication was apparent on following passage to ST cells as evident by cytopathic effects and measurement of viral titer using the hemagglutination assay (640 HA units/mL). Full genome sequencing verified the rescued virus (IAV/HA:IDV HEF) contained all segments derived from pH1N1 with the exception of the HA gene which was replaced with the chimeric genome segment containing the HEF gene (SEQ ID NO:4) of D/OK. This virus is consequently an IAV/IDV chimera as it lacks the IAV HA gene and rather uses the HEF from D/OK for receptor attachment.

### Example 4

As the HEF protein encodes both receptor binding and destruction activities, we next sought to replace the NA gene (SEQ ID NO:5) with those of heterologous genes encoding pathogenic antigens. Similar to Example 1, we constructed a plasmid using the pHW2000 backbone and NA packaging sequences (see SEQ ID NO:3). A DNA construct was designed in silico such that it contained 130 and 185 nucleotides of the NA coding region (3′ and 5′ ends, respectively) flanking the PRRS nucleocapsid gene (SEQ ID NO:). Similar to pHA_{packaging}, NotI and SbfI restriction sites were included at the junction of the 5′ and 3′ packaging sequences and the PRRS N gene, respectively. The synthetic DNA fragment was amplified with NA segment specific primers from Hoffmann et al. and digested with BsaI and ligated into a similarly digested pHW2000 to create pNA-PRRS-N (SEQ ID NO:7). The sequence of the plasmid was confirmed by Sanger sequencing. Plasmids derived from pHW2000 containing IAV pH1N1 PB2, PB1, PA, NP, M and NS genomic segments were co-transfected into a co-culture of 293T/MDCK cells along with pD/OK HEF (SEQ ID NO:4) and pNA-PRRS-N (SEQ ID NO:7). Viral replication was apparent on following passage to ST cells as evident by cytopathic effects and measurement of viral titer using the hemagglutination assay (640 HA units/mL).

The rescued virus, IAV/HA:IDV HEF/NA:PRRS N, was next used to infect ST cells. At 24 hours, the cells were fixed with 80% acetone and stained with a monoclonal antibody (FITC conjugate) generated against the PRRS N protein (SDOW17, RTI Technologies) at a dilution of 1:50. Direct immunofluorescence microscopy revealed green fluorescence for infected cells and no fluorescence for uninfected cells or infected cells not stained with SDOW17. These results demonstrate that the chimera virus bearing the IDV HEF functionally complements the native HA and NA protein activities resulting in viable virus. Additionally, these results demonstrate that the NA segment can be used for heterologous antigen expression as verified by IFA.

### Example 5

To further test the ability of the IAV/IDV chimera virus platform to express heterologous proteins, we cloned the marker protein green fluorescent protein from jellyfish into our plasmid bearing NA packaging sequences (SEQ ID NO:3). pNA-PRRS-N was digested with NotI and SbfI and the plasmid backbone was isolated from an agarose gel. The coding sequence for GFP (residues 139-858 of SEQ ID NO:10) was obtained from Genbank and synthesized with NotI and SbfI restriction sites immediately upstream and downstream of the start and stop codons, respectively. The synthetic DNA construct was digested with NotI and SbfI and ligated into the gel purified backbone from digested pNA-PRRS-N and its sequence (pNA-GFP) was verified by Sanger sequencing (SEQ ID NO:10). Plasmids derived from pHW2000 containing IAV pH1N1 PB2, PB1, PA, NP, M and NS genomic segments were co-transfected into a co-culture of 293T/MDCK cells along with pD/OK HEF (SEQ ID NO:4) and pNA-GFP (SEQ ID NO:10).

Viral replication was apparent on following passage to ST cells as evident by cytopathic effects and measurement of viral titer using the hemagglutination assay (640 HA units/mL). The rescued virus, IAV/HA:IDV HEF/NA:GFP, was next used to infect ST cells. Cells were observed directly with a fluorescent microscope and a high percentage fluoresced, indicating successful expression of GFP by the IAV/IDV chimera virus IAV/HA:IDV/NA:GFP.

### Example 6

To further test the ability of the IAV/IDV chimera virus platform to express heterologous proteins, we cloned a portion (nucleotides 1-2257) of the spike gene (S1) of porcine epidemic diarrhea virus into our plasmid bearing NA packaging sequences. pNA-PRRS-N was digested with NotI and SbfI and the plasmid backbone was isolated from an agarose gel. The coding sequence for PEDV S1 was obtained from Genbank and synthesized with NotI and SbfI restriction sites immediately upstream and downstream of the start and stop codons, respectively. The synthetic DNA construct was digested with NotI and SbfI and ligated into the gel purified backbone from digested pNA-PRRS-N and its sequence (pNA-PEDV S1) was verified by Sanger sequencing. Plasmids derived from pHW2000 containing IAV pH1N1 PB2, PB1, PA, NP, M and NS genomic segments were co-transfected into a co-culture of 293T/MDCK cells along with pD/OK HEF (SEQ ID NO:4) and pNA-PEDV S1 (SEQ ID NO:8).

Viral replication was apparent on following passage to ST cells as evident by cytopathic effects and measurement of viral titer using the hemagglutination assay (640 HA units/mL). The rescued virus, IAV/HA:IDV HEF/NA:PEDV S1, was next used to infect ST cells. At 24 hours the cells were fixed with 80% acetone and stained with either a monoclonal antibody or polyclonal antisera generated against PEDV S1 (Eric Nelson, South Dakota State University) at a dilution of 1:50. Cells were subsequently treated with secondary anti-mouse or anti-rabbit antibodies (FITC conjugated) and observed by fluorescent microscopy. Both antibodies revealed foci of infected cells with green fluorescence. Uninfected cells failed to show fluorescence as did infected cells not treated with primary antibody. These results demonstrate expression of PEDV SI that is recognized by both monoclonal and polyclonal antibodies.

### Example 7

To further test the ability of the IAV/IDV chimera virus platform to express heterologous proteins, we cloned the capsid gene of porcine circovirus 2 into our plasmid bearing NA packaging sequences (SEQ ID NO:3). pNA-PRRS-N was digested with NotI and SbfI and the plasmid backbone was isolated from an agarose gel. The coding sequence for PCV2 capsid was obtained from Genbank and synthesized with NotI and SbfI restriction sites immediately upstream and downstream of the start and stop codons, respectively. The synthetic DNA construct was digested with NotI and SbfI and ligated into the gel purified backbone from digested pNA-PRRS-N and its sequence (pNA-PCV2 cap) was verified by Sanger sequencing. Plasmids derived from pHW2000 containing IAV pH1N1 PB2, PB1, PA, NP, M and NS genomic segments were co-transfected into a co-culture of 293T/MDCK cells along with pD/OK HEF (SEQ ID NO:4) and pNA-PCV2 cap (SEQ ID NO:9).

Viral replication was apparent on following passage to ST cells as evident by cytopathic effects and measurement of viral titer using the hemagglutination assay (640 HA units/mL). The rescued virus, IAV/HA:IDV HEF/NA:PCV2 cap, was next used to infect ST cells. At 24 hours the cells were fixed with 80% acetone and stained with a FITC conjugated antibody generated against PCV2 (VMRD) at a dilution of 1:50. Cells were subsequently observed by fluorescent microscopy and revealed foci of infected cells with green fluorescence localized in the cellular nuclei. Uninfected cells failed to show fluorescence as did infected cells not treated with antibody. Nuclear expression was expected as the capsid gene contains a N-terminal nuclear localization sequence. These results demonstrate expression of PCV2 capsid that is recognized by polyclonal antibodies generated against PCV2.

### Example 8

An eight-segmented influenza chimeric virus was synthesized and rescued containing seven segments from influenza A (A/California/04/2009 H1N1): M, NS, PB1, PB2, PA, NP, and NA, and one segment from influenza D (D/swine/OK/1334/2011 (D/OK)): HEF, as described above. A virus with elastase-dependent mutations in the HEF trypsin cleavage site was also rescued. Protection to a heterosubtypic virus was evaluated in pigs vaccinated with the chimera vaccines.

### Materials and Methods:

Cells and viruses. Madin-Darby canine kidney (MDCK) cells, swine testicle (ST) cells, and 293T (human embryonic kidney) cells were all maintained in minimal essential media (MEM) supplemented with L-glutamine and 5% fetal bovine serum. Challenge virus A/swine/MN/2073/2008 (MN08) was obtained from South Dakota State University (SDSU), with viral titer determined by titration on MDCK cells.

Plasmids. All eight cDNA segments of A/CA/04/2009 were cloned into the vector pHW2000 (gift of Dr. Wenjun Ma). D/OK HEF pHW2000 plasmid was a kind gift of Dr. Feng Li. PCR was performed using primers to amplify D/OK HEF and add 5′-NotI and 3′-Sbf restriction sites. The IAV HA packaging sequences were synthesized as a DNA fragment that contained NotI and SbfI restriction sites immediately downstream and upstream of the 5′ and 3′ packaging sequences, respectively, separated by an 18bp linker. The synthetic DNA was used as template for PCR using universal HA segment-specific primers that introduced BsmBI restriction sites at the product termini. The PCR product was purified using a Qiagen PCR cleanup column and then digested with BsmBI and ligated in to the pHW2000 plasmid to generate pHW-HApack. The D/OK HEF PCR product was digested with NotI and SbfI and ligated into a similarly digested pHW-HApack to yield pHW-HA.HEF.HA. Additionally, the D/OK HEF segment was mutagenized using the QuikChange II SL Site-Directed Mutagenesis kit (Agilent Technologies) to introduce the elastase-dependent motifs as described by Masic et al. 2009. Reverse genetics-generated elastase-dependent swine influenza viruses are attenuated in pigs. J. Gen. Virol. 90: 375-385.

Generation of viruses by reverse genetics. Eight-plasmid reverse genetic viruses were generated. Briefly, 293T and MDCK cells were co-cultured at equal density in a six-well plate in Opti-MEM (Invitrogen) with 5% fetal bovine sera and incubated overnight. Prior to transfection, the wells were rinsed once with phosphate buffered saline (PBS). 100 ng/uL of each of the eight plasmid constructs were transfected using lipofectamine transfection reagent (Life Technologies). After a six hour incubation, the transfection mixture was replaced with 1mL of Opti-MEM plus penicillin/streptomycin solution (HyClone). At 24 hours post transfection, 1mL of MEM containing TPCK-treated trypsin was added to each well. At 48 hours post transfection, supernatants were harvested and passaged to ST cells in MEM. The two rescued viruses were D/OK-pN1 and D/OKelast-pNl. These viruses contained seven segments derived from A/California/04/2009 (PB2, PB1, PA, NP, NA, M and NS) and either the native HEF gene from D/OK (D/OK-pN1) or a HEF gene with an elastase dependent cleavage site (D/OKelast-pN1). The D/OKelast-pN1 virus received a supplement of 1 μg/mL of elastase from porcine pancreas (Sigma). The elastase mutations were confirmed by sequence analysis.

Elastase-dependent in vitro growth kinetics. To determine if the elastase mutation resulted in elastase dependence in vitro, a growth study was performed on ST cells. The D/OK-pN1 and D/OKelast-pN1 viruses were titrated and T75 flasks of confluent STs were infected with 2.5 log₁₀ tissue culture infective dose (TCID₅₀) per mL of D/OK-pN1, D/OKelast-pN1 with elastase supplemented at 1 ug/mL, and D/OKelast-pN1 without elastase supplementation. Two samples were collected from each flask at the start of the experiment (T0), and at 24, 48, 72, and 96 hours post infection. The duplicate samples for each time point of D/OK-pN1 and D/OKelast-pN1 grown without supplemented elastase were titrated in MEM without elastase. The samples of D/OKelast-pN1 grown with supplemental elastase were titrated in MEM with elastase.

Vaccination/challenge pig experiment. Both D/OK-pN1 and D/OKelast-pN1 were passaged on ST cells and titrated for use in the vaccination/challenge study. Twenty-four 3-week-old pigs were obtained from a high health herd. They were confirmed SIV seronegative by IDEXX SIV ELISA and allowed one week to acclimate prior to vaccination. Pigs were divided into three groups; eight pigs in group A received 6.0 log₁₀ TCID₅₀ of D/OK-pN1 intranasally, eight pigs in group B received 6.0 log₁₀ TCID₅₀ of D/OKelast-pN1 intranasally, and group C consisted of 8 pigs that received mock vaccinations. Group C was housed with the group B animals. All animals were re-vaccinated at 21 days. At 35 days all animals, except two from the group C control group, were challenged intranasally with 6.0 log₁₀ TCID₅₀ of MN08.

Clinical observation and sampling. Nasal swabs were collected at day 0 (pre-vaccination), day 1, 3, and 5 post initial vaccination. Nasal swabs were collected at days 0, 1, 3, and 5 post challenge (days 35, 36, 38, and 40) to monitor shedding. All swabs were placed in MEM and frozen at −80° C. until ready for further testing. Animals were monitored daily for signs of illness. Three animals from groups A and B, and two animals from group C were sacrificed at day 3 and day 5 post-challenge and lungs and bronchial alveolar lavage fluids (BALF) samples were collected and frozen. BALF was collected by adding 50 mL of DMEM to the trachea from lungs collected in toto and gently palpating the lungs before pouring off fluid. Lung tissues were collected from the right cardiac lobe and analyzed by titration and histopathology. Serum was collected at day 0, 21, and 35.

Virus shedding from nasal swabs, lungs, and BALFs. Nasal swabs collected on day 0, 1, 3, and 5 post-vaccination were tested by quantitative real-time reverse transcriptase PCR (qRT-PCR). RNA was extracted using the MagMax RNA isolation kit. qRT-PCR was performed according to the approved National Animal Health Laboratory Network universal IAV protocol employed at KSVDL. Nasal swabs from day 0, 1, 3, and 5 post challenge, as well as the BALF samples were titrated on ST cells. Samples were thawed and vortexed, then centrifuged at 2000 RPM for 5 minutes. Lung homogenates were prepared in MEM with antibiotics using a stomacher. Two mLs of homogenate were centrifuged at 2000 RPM for ten minutes to pellet debris. Supernatants were then serially diluted and plated (four replicates) onto confluent ST cells in 96-well plates. Plates were monitored for cytopathic effects (CPE) and confirmed via hemagglutination assay using Turkey red blood cells. Titers were calculated by Reed & Muench method.

### Serology

IgA ELISA. Enzyme-linked immunosorbent assay (ELISA) was performed on BALF samples. Briefly, vaccine virus D/OK-pN1 and challenge virus MN08 were ultracentrifuged at 27,000 RPM for four hours through a 20% sucrose cushion and coated overnight onto ELISA plates separately, at 100 HA units/50 uL. The plates were blocked for one hour at room temperature using starting block (Fisher). Plates were rinsed three times with phosphate buffered saline (PBS) with 0.05% Tween 20 (PBST). BALF samples were diluted 1:2 with 10mM dithiothreitol (DTT) to disrupt mucus, and incubated for one hour at 37° C. before running the assay. Samples were then diluted 1:2 in PBS to a final dilution of 1:4. Fifty microliters of each DTT treated BALF sample was added to duplicate wells on each antigen-coated plate. A heat-inactivated known IAV negative serum sample was used as a negative control serum (diluted 1:4 in PBS). Plates were incubated for one hour at room temperature then washed three times with PBST. Fifty microliters of goat anti-pig IgA HRP secondary antibody (Abcam) was applied at 1:1500 (diluted in starting block) and incubated for one hour at room temperature. Plates were washed three times with PBST and 50 uL of ABTS ELISA substrate was added and incubated at room temperature approximately eight minutes. The reaction was stopped by adding 50 uL stop solution (KPL). The optical density (OD) was read at 405nm using an automated plate reader. OD values were averaged for each sample.

Hemagglutination inhibition assay. Hemagglutination inhibition (HI) assay was used to detect antibodies to vaccine virus D/OK-pN1 and challenge virus MN08. The test was performed using Turkey red blood cells, according to the WHO manual. Samples assayed included serum from day 0 (pre-initial vaccination), day 21 (pre-second vaccination), and day 35 (pre-challenge).

Serum neutralization assay. Serum samples from group A at day 21 (day of second vaccination) and day 35 (day of challenge) were assayed for neutralizing antibodies against the challenge and vaccine strain viruses. A 1:4 dilution of heat-inactivated sera was added to MEM and serially two-fold diluted down 96-well plates. An equal volume of antigen, ranging from 40-80 virus particles per well, was added to the plates and they were incubated for one hour at 37° C. The virus/serum mixture was then transferred to a monolayer of ST cells and incubated for four days. Antibody titers were determined by the last well protected from cytopathic effects. For statistical analysis, geometric mean titers were calculated from loge transformed titers.

Enzyme-linked lectin-based neuraminidase inhibition assay. A neuraminidase inhibition (NI) assay was performed. Briefly, 96-well plates were coated with 25ug/mL of fetuin (Sigma) and incubated overnight. Day 35 serum samples were heat inactivated at 56° C. for one hour. Sixty microliters of sample diluent (PBS, 1% bovine serum albumin, and 0.5% Tween 20) was added to columns 2-11 of an uncoated 96-well plate. Column one received 114 uL of sample diluent and 6 uL of heat inactivated sera, resulting in a 1:20 dilution. Next, 63 uL from column one were serially two-fold diluted through column 11. The top three wells of column 12 received only sample diluent. The last five wells of column 12 served as a back-titer control for serially diluted virus. Fifty microliters of these serum dilutions were transferred to fetuin plates that had been washed three times in PBST. Fifty microliters of challenge virus, which contains an antigenically-mismatched HA from our vaccine strain HEF, were added at a pre-determined dilution. Plates were sealed and incubated overnight at 37° C. The plates were then rinsed six times in PBST. One-hundred microliters of a 1:1000 dilution of peroxidase-labeled peanut agglutinin (PNA-HRPO) (Sigma) was added to the plates and incubated for two hours at room temperature, in the dark. The plates were washed again as before. One-hundred microliters of o-phenylenediamine dihydrochloride (OPD) (Sigma), dissolved in citrate buffer (Sigma), were added to the plates. Plates were incubated in the dark for ten minutes before adding 100 uL of 1N sulfuric acid stop solution. Optical densities were read on a plate reader at 490nm.

Histopathology and Immunohistochemistry. Gross lung lesion scores were determined. The lung samples from the right cardiac lobe were submitted for microscopic lung lesion evaluation. Lungs were examined for airway epithelial necrosis and loss, airway inflammation, airway lymphocytic cuffing, and interstitial pneumonia. They were scored on a scale of 0 to 4 and reported as an average, as described by Henningson et al. 2015. Comparative virulence of wild-type H1N1 pdm09 influenza A isolates in swine. Vet. Microbiol. 176(1-2): 40-9. Immunohistochemical (IHC) examination scored the lungs based on airway and interstitium IHC and reported as averages. The pathologist was blinded to the identity of animals and groups the lung tissues originated from.

### Results

Statistical analysis was performed using JMP Version 12, SAS Institute Inc.

In vitro growth kinetics. The D/OK-pN1 and D/OKelast-pN1 viruses were successfully rescued via reverse genetics and further propagated on ST cells. The duplicate samples from each time-point of each virus were titrated and their average titer was determined by Reed-Muench. D/OK-pN1 reached a titer of 6.7 log₁₀ TCID₅₀. D/OKelast-pN1 grown in the presence of elastase supplement and titrated with elastase supplement reached 7.7 log₁₀ TCID₅₀. The D/OKelast-pN1 grown without elastase supplementation failed to grow (FIG. 4).

Viral Shedding from nasal swabs, lungs, and BALF. Attempts to titrate the nasal swabs following the initial vaccination were unsuccessful due to non-influenza cytopathic effects. qRT-PCR was performed on these samples and virus was only detected in group A vaccinates, whereas all group B elastase vaccinates were negative for SIV qRT-PCR (Table 1).

TABLE 1

| Group | Day 1 | Day 3 | Day 5 | Day 7 |
|---|---|---|---|---|
| A N = 8 | 32.41 | 31.5 | 31.13 | 35.49 |
| B N = 8 | >37 | >37 | >37 | >37 |
| C N = 8 | >37 | >37 | >37 | >37 |

Average CTs from qPCR for each group post-vaccination. >37 is considered negative

Post-challenge viral titers in the BALFs, as determined by titration, yielded results ranging from 0 to 6.95 log₁₀ TCID₅₀. Virus titers in BALFs of the group A and B animals showed no statistically significant difference in titers at day 3 post challenge when compared to the controls. Animals sacrificed at 5 days post challenge from group A had no detectable IAV in their BALFs, which was statistically different from group B and controls (Table 2).

TABLE 2

| | | Lung | BALF | IgA ELISA | IgA ELISA |
|---|---|---|---|---|---|
| | | titers post | titers post | OD Vaccine | OD Challenge |
| Group | | challenge | challenge | Strain | Strain |
| A: 6.0 TCID₅₀ | Mean | 4.62^{A} | 5.7^{A} | 0.34^{B} | 0.20^{B} |
| of D/OK-pN1 | | | | | |
| Day 3 Post | Std. Deviation | 1.18 | 0.25 | 0.18 | 0.09 |
| Challenge | | | | | |
| N = 3 | Std. Error | 0.68 | 0.14 | 0.10 | 0.05 |
| B: 6.0 TCID₅₀ | Mean | 5.03^{A} | 5.53^{A} | 0.12^{B} | 0.11^{B} |
| of D/OKelast-pN1 | | | | | |
| Day 3 Post | Std. Deviation | 0.52 | 0.29 | 0.01 | 0.01 |
| Challenge | | | | | |
| N = 3 | Std. Error | 0.30 | 0.17 | 0.01 | 0.00 |
| C: Mock Vx | Mean | 5.7^{A} | 6.7^{A} | 0.13^{B} | 0.13^{B} |
| Day 3 Post | Std. Deviation | 0.35 | 0.35 | 0.00 | 0.03 |
| Challenge | | | | | |
| N = 2 | Std. Error | 0.25 | 0.25 | 0.00 | 0.02 |
| A: 6.0 TCID₅₀ | Mean | 0.00*^{B} | 0.00*^{B} | 0.92*^{A} | 0.74^{*A} |
| of D/OK-pN1 | | | | | |
| Day 5 Post | Std. Deviation | 0.00 | 0.00 | 0.36 | 0.32 |
| Challenge | | | | | |
| N = 3 | Std. Error | 0.00 | 0.00 | 0.21 | 0.19 |
| B: 6.0 TCID₅₀ | Mean | 4.12^{A} | 3.53^{A,B} | 0.14^{B} | 0.100⁸ |
| of D/OKelast-pN1 | | | | | |
| Day 5 Post | Std. Deviation | 1.01 | 2.74 | 0.04 | 0.04 |
| Challenge | | | | | |
| N = 3 | Std. Error | 0.58 | 1.58 | 0.02 | 0.02 |
| C: Mock Vx | Mean | 4.7^{A} | 4.7^{A} | 0.10^{B} | 0.10^{B} |
| Day 5 Post | Std. Deviation | 0.71 | 0.35 | 0.01 | 0.00 |
| Challenge | | | | | |
| N = 2 | Std. Error | 0.5 | 0.25 | 0.01 | 0.00 |

^{A,B}Tukey HSD lists different letters between groups whose means that are statistically significant. Those with same letters means no significant difference among their means.
^{*}The mean difference is significant at the 0.05 level compared to the control group.

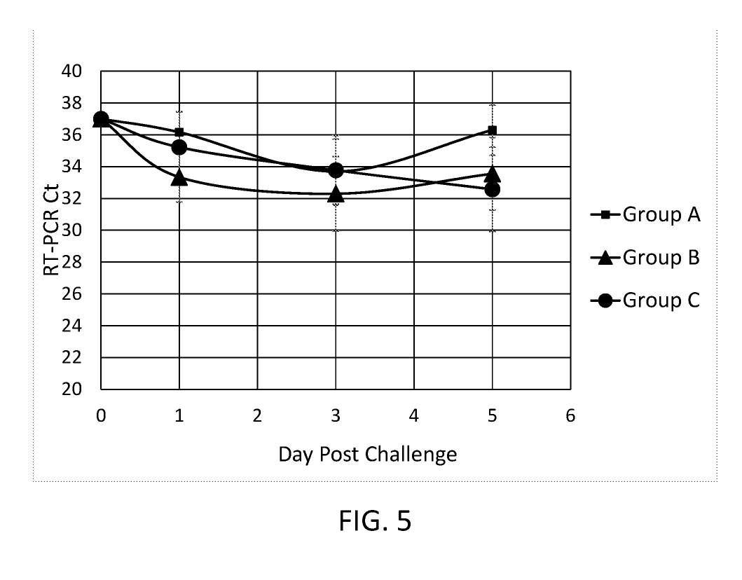

There was no significant differences for BALF titers in the group B vaccine group from the controls. The titration of the lung samples yielded results similar to BALF, with group A animals showing a statistically significant decrease in viral shedding at day 5 post challenge compared to group B and controls, with no influenza virus detected in the lung. Nasal swabs collected at day of challenge, day 1, 3, and 5 post challenge were also titrated and ran on qRT-PCR. All nasal swabs were less than 1.5 log₁₀ TCID₅₀ and the qRT-PCR values ranged from 30-37 (Ct>37 considered negative) (FIG. 5).

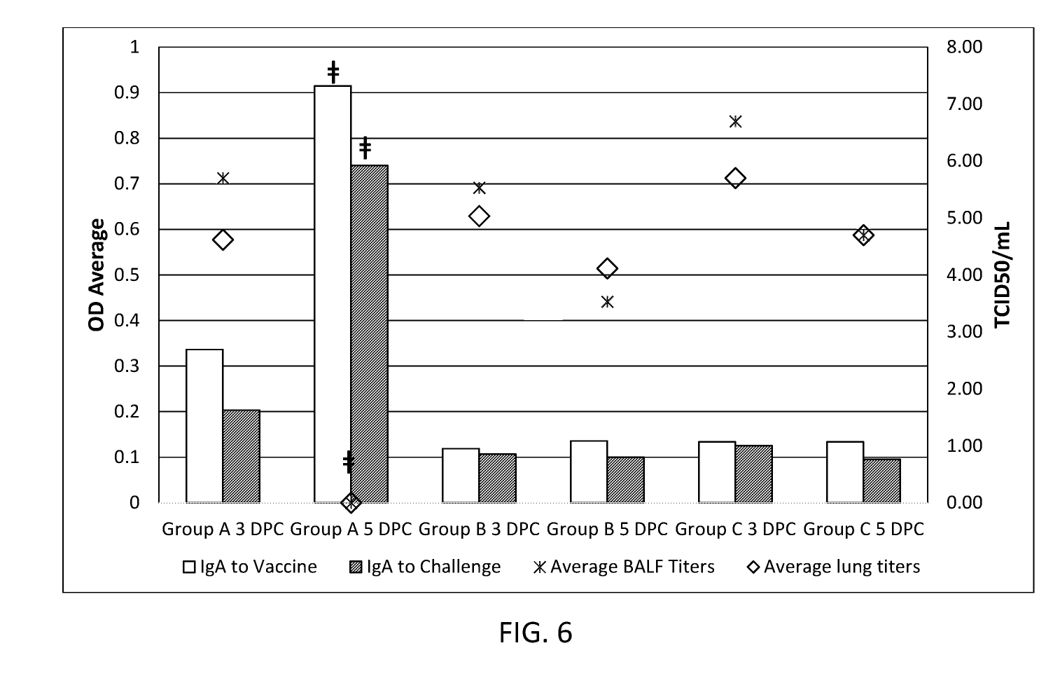

Serology to Vaccine Strain. The results of the IgA ELISA on BALF samples showed there was statistically higher IgA OD values for the vaccine strain in the group A pigs at 5 days post challenge when compared to group B and controls (Table 2) (FIG. 6). Group B IgA ODs were not statistically different from the control group. The vaccine strain showed statistically significant HI antibody titers in group A day 21 and 35 samples compared to the day 0 sera, with log₂ transformed geometric mean titers ranging of 6.94, 7.15, and 0, respectively. The control non-vaccinated animals as well as group B animals were negative for antibody to vaccine virus via HI. The serum neutralization assay on the group A serum showed statistically significant higher antibody titers to the vaccine strain at day 35, loge transformed geometric mean titer of 7.31, compared to the challenge virus at day 35, (4.67). The non-vaccinated controls had average antibody titers of 2.0 and 3.0 to vaccine and challenge viruses, respectively. Serum neutralizations assays were not performed on group B vaccinates. No titers were measured in any group by the NI assay (data not shown).

Serology to Challenge Strain. The results of the IgA ELISA on BALF samples showed there was statistically higher IgA OD values for the challenge strain in the group A pigs at 5 days post challenge when compared to group B and controls (Table 2) (FIG. 6). Group B IgA ODs were not statistically different from the control group. The HI assay showed no antibody titer to the challenge virus was present in the serum samples. Serum neutralization assay showed an increase compared to the control animals, but it was not a statistically significant increase. No titers were measured in any group by the NI assay (data not shown).

Histopathology and IHC. Gross lesions were more evident in the 5 days post challenge lungs than the 3 days post challenge lungs, in all groups. The histopathological exam, as well as IHC, showed no statistically significant differences between vaccinate groups or date of sacrifice when compared to controls. The histopathological exam average scores ranged from 0 to 2.5. The IHC average scores ranged from 0 to 3.5. The control animals showed no signs of influenza infection.

### Discussion

Influenza virus continues to be an important pathogen in the swine industry. Morbidity rates can approach 100%, which results in economic losses to the producer. We rationalized that a live chimeric influenza A/D virus would replicate in swine and induce a protective immune response to conserved viral antigens. Serological results from vaccinated pigs demonstrated that the vaccine is immunogenic. Despite a lack of measurable HI and NI titer to the challenge virus and SN titers not significantly different than the non-vaccinated controls, pigs vaccinated with live chimeric influenza A/D virus were able to clear virus from their lungs by day 5 post challenge. This represents an approximate 5 log₁₀ TCID₅₀/mL reduction in lung titer as compared to non-vaccinated controls. By decreasing the amount of time pigs are infected with influenza virus, the economic loss to the producer can be minimized and virus transmission can be decreased.

Conventional inactivated vaccines rely on a strong humoral immune response to the HA. As HA is the most genetically diverse IAV protein, limited cross-protection is observed between viruses with genetically divergent HA' s. Other modified live IAV vaccines have shown a cell-mediated immune response may play a role in heterologous protection, as they only induce homologous HI titers yet demonstrate heterologous protection. Another issue with current inactivated vaccines is their inability to be used in young pigs due to maternally-derived antibody (MDA) interference from the vertical transfer of humoral IgG antibodies from vaccinated sows to piglets. As only 9.5% of pigs were seropositive for IDV and the GMT HI titers for positive animals was approximately 20, pigs are largely naive to IDV. As the chimera IAV/IDV virus displays HEF on the virion surface, it is unlikely that maternally-derived anti-IAV antibodies will cross react with the chimera vaccine.

The D/OKelast-pN1 vaccine failed to induce an immune response in this study. The elastase mutation may have too severely attenuated the virus to be immunogenic at 6.0 log₁₀ TCID₅₀. The PCR results post-vaccination showed no shedding from the elastase groups, consistent with the in vitro growth kinetics where elastase mutants failed to grow without elastase supplementation.

Nasal swabs were collected from all pigs on the day of challenge and on days 1, 3, 5 and 7 post challenge. The post-challenge nasal swabs had no detectable live virus by titration, detected only low levels of virus by qRT-PCR, and CTs were in the 30-37 range for all groups.

Here we utilized the HEF gene of IDV to attenuate IAV. As the virus receptor binding protein is critical for cell tropism, pathogenicity and immunity, it's likely that the HEF properties that differ from HA contribute to its attenuation in swine as compared to parental IAV. This could be due to the HEF utilizing Neu5,9Ac2 as a receptor instead of the abundant α-2,3 and α-2,6 sialic acids that IAV binds to in the respiratory tract of swine. The chimeric viruses in this experiment encoded HEF in the HA genomic segment. Expression of the HEF from the NA segment could prevent reassortment in pigs and generation of wild type and vaccine reassortants. The protection conferred by vaccination with the chimeric virus is independent of HA and NA. The ability of this chimeric vaccine to stimulate the mucosal immune system and rapidly clear virus from the lungs offers new technology for swine influenza vaccines and their use in young pigs.

1. A chimeric influenza virus A, comprising a backbone of viral genomic segments derived from influenza A, and expressing a heterologous surface protein, wherein said heterologous surface proteins is influenza D hemagglutinin esterase fusion (HEF) protein.

2. The chimeric influenza virus of claim 1, wherein said HEF protein comprises SEQ ID NO:1.

3. The chimeric influenza virus of claim 1, comprising an internal chimeric viral genome segment encoding for said HEF protein.

4. The chimeric influenza virus of claim 3, wherein said internal chimeric viral genome segment is a chimeric Hemagglutinin (HA) or Neuraminidase (NA) polynucleotide viral segment encoding a protein open reading frame for said HEF protein, and comprising noncoding regions and viral packaging sequences derived from the native HA or NA polynucleotide segment of said influenza A.

5. The chimeric influenza virus of claim 4, wherein said internal chimeric viral genome segment is a chimeric HA polynucleotide viral segment, said chimeric virus comprising an internal viral genome segment encoding for native influenza A NA.

6. The chimeric influenza virus of claim 4, wherein said chimeric HA polynucleotide viral segment comprises an HA packaging construct according to SEQ ID NO:2, where n indicates the location where the coding sequence encoding for the protein open reading frame is inserted.

7. The chimeric influenza virus of claim 4, wherein said chimeric HA polynucleotide viral segment comprises SEQ ID NO:4.

8. The chimeric influenza virus of claim 4, wherein said chimeric HA polynucleotide viral segment encodes for a protein according to SEQ ID NO:1.

9. The chimeric influenza virus of claim 1, wherein said backbone consists of six viral segments derived from: SEQ ID NO:11 (PB1), SEQ ID NO:13 (PB2), SEQ ID NO:15 (PA), SEQ ID NO:17 (NP), SEQ ID NO:19 (M), and SEQ ID NO:22 (NS).

10. The chimeric influenza virus of claim 1, wherein said backbone segments encode for one or more viral proteins selected from the group consisting of SEQ ID NO:12 (PB1), SEQ ID NO:14 (PB2), SEQ ID NO:16 (PA), SEQ ID NO:18 (NP), SEQ ID NO:20 (M1), SEQ ID NO:21 (M2), SEQ ID NO:23 (NS1), and SEQ ID NO:24 (NS2).

11. The chimeric influenza virus of claim 1, said chimeric virus further expressing a heterologous antigenic component of a target pathogen.

12. The chimeric influenza virus of claim 11, wherein said target pathogen is selected from the group consisting of Porcine reproductive and respiratory syndrome virus (PRRSV), Porcine epidemic diarrhea virus (PEDV), Porcine Circovirus Type 2 (PCV2), Porcine deltacoronavirus (PDCoV), Bovine coronavirus (BCV), and Bovine respiratory syncytial virus (BRSV).

13. The chimeric influenza virus of claim 11, comprising an internal chimeric viral genome segment encoding for said heterologous antigenic component, wherein said internal chimeric viral genome segment is a chimeric HA or NA polynucleotide viral segment encoding a protein open reading frame for said heterologous antigenic component, and comprising noncoding regions and viral packaging sequences derived from the native HA or NA polynucleotide segment of said influenza A.

14. The chimeric influenza virus of claim 13, wherein said internal chimeric viral genome segment is a chimeric NA polynucleotide viral segment.

15. The chimeric influenza virus of claim 14, wherein said chimeric NA polynucleotide viral segment comprises an NA packaging construct according to SEQ ID NO:3, where n indicates the location where the coding sequence encoding for the antigenic component protein open reading frame is inserted.

16. The chimeric influenza virus of claim 13, wherein said internal chimeric viral genome segment is a chimeric NA polynucleotide viral segment comprising SEQ ID NO:7, SEQ ID NO:8, or SEQ ID NO:9.

17.-19. (canceled)

20. The chimeric influenza virus of claim 1, said chimeric virus encoding a fluorescent marker, wherein said chimeric virus comprises an internal chimeric NA polynucleotide viral segment encoding for said fluorescent marker and comprising SEQ ID NO:10.

21. A vaccine composition useful for inducing an immune response against viral infection, comprising:
a therapeutically-effective amount of chimeric influenza A virus according to claim 1 dispersed in a pharmaceutically-acceptable carrier; and
optionally, one or more adjuvants, active agents, preservatives, buffering agents, or salts dispersed in said carrier.

22. A method of vaccinating a subject to induce an immune response against viral infection, said method comprising administering a vaccine composition according to claim 21 to said subject.

23. The method of claim 22, wherein said administering is selected from the group consisting of: (a) injecting said vaccine composition intramuscularly, subcutaneously, intradermally, or intravenously using a needle and syringe, or a needleless injection device; and (b) intranasal administration as drops, large particle aerosol, or a spray.

24.-29. (canceled)

30. A synthetic cDNA comprising SEQ ID NO:4 encoding for HEF surface protein useful for generating chimeric influenza A viruses.

31. A synthetic cDNA useful for generating chimeric influenza A viruses comprising noncoding regions and viral packaging sequences derived from a native HA polynucleotide segment of influenza A, and a heterologous polynucleotide sequence encoding for an open reading frame for one or more heterologous proteins, said cDNA comprising SEQ ID NO:2, where n indicates the location where the heterologous polynucleotide sequence is inserted.

32. A synthetic cDNA useful for generating chimeric influenza A viruses comprising noncoding regions and viral packaging sequences derived from a native NA polynucleotide segment of influenza A, and a heterologous polynucleotide sequence encoding for an open reading frame for one or more heterologous proteins, said cDNA comprising SEQ ID NO:3, where n indicates the location where the heterologous polynucleotide sequence is inserted.
